(19) United States
(12) Patent Application Publication
(10) Pub. No.: US 2024/0301491 A1
(43) Pub. Date: Sep. 12, 2024

(54) DEVICES, METHODS AND KITS FOR BIOLOGICAL SAMPLE CAPTURE AND PROCESSING

(71) Applicant: VosBio, Inc., San Jose, CA (US)

(72) Inventors: Douglas T. GJERDE, Saratoga, CA (US); Daniel Robert BOLLINGER, San Jose, CA (US)

(21) Appl. No.: 18/180,400

(22) Filed: Mar. 8, 2023

(51) Int. Cl. C12Q 1/6881; A61B 5/097; C12Q 1/6844

(52) U.S. Cl. CPC C12Q 1/6881; A61B 5/097; C12Q 1/6844

(57) ABSTRACT

Breath liquid particles and vapor are captured in a device presenting a surface and chamber space that condenses or freezes the vapor and aerosol particulates. One or more breaths are exhaled through the device. Capture can be performed on the freezing surface immobilizing water upon contact. The chamber space within the device may freeze or condense liquid breath particles and vapor to collect them. After collection, the liquid is gathered and collected either by draining, scraping, pushing, or centrifugal force. The liquid may be collected and combined with a sample preparation reagent such as a virus lysing reagent, an internal standard, etc. After collection, the sample is separated into a plurality of independently analyzable units for detection and quantification of a target. Analysis may be performed by PCR, qPCR RT-PCR, RT-qPCR, LAMP, RPA or any nucleic acid detection method, lateral antigen mass spectrometry, spectrophotometry or any analytical tool or method. Nucleic acid amplification reagents may contain a lysing reagent such as acetonitrile.

### FIELD OF THE INVENTION

The present invention relates to devices, methods and kits for capturing and processing biological samples from breath, in particular for capturing biological and organic samples that are liquid, particulate or vapor.

### BACKGROUND OF THE INVENTION

Sample collection for the detection of virus, bacteria and other organic materials is sometimes difficult, especially for children. A breath sample is less intrusive and a desirable way to sample for virus. In principle if someone is shedding and exhaling virus, the person is infectious and may spread the viral disease. However, liquid from breath is not easy to collect. Current breath liquid collection is an arduous process taking time and effort of an individual breathing into an apparatus. In addition, breath generally contains a lower viral load and bacterial load than a saliva or nostril swab sample.

In current technology, there are limitations in capturing exhaled breath quickly and efficiently. For purposes of widespread, rapid testing for infectiousness, breath needs to be collected rapidly from large numbers of people. Many conventional systems recommend 5-10 minutes of breath to be collected prior to analysis, typically yielding a sample of 1 mL of liquid. Conventional systems may also require a cooling sleeve to be cooled in a freezer several times to provide cooling, making those systems impractical for large-scale use. Other devices for breath collection utilize similar or longer timelines, requiring at least 10 minutes each.

U.S. Pat. No. 7,118,537 describes a device for condensing samples of fluid from breath in which a sleeve surrounding a collection tube may be chilled, e.g., in a home refrigerator, to improve efficiency of collection.

Many infectious diseases are spread by infected individuals shedding and exhaling viruses through their breath. In many cases, the pathogens are contained in exhaled airborne liquid particles. Liquid particles containing the shed pathogens may remain airborne for extended periods, thus increasing the likely hood of pathogens contacting and infecting multiple individuals in proximity to the infectious individual.

The degree to which an individual may be infectious may depend on the number of pathogens an infectious individual is shedding and exhaling. The total number of pathogens exhaled and the rate of pathogens exhaled are both important.

An infectious individual is likely to shed and spread pathogens before any symptoms are felt or shown by the infectious individual.

Accordingly, there exists a need to capture the liquid particles and vapor quickly as 1 min or less from a breath sample for viral, bacterial, biological and chemical analysis to provide a rapid indication of the extent to which pathogen are being shed. There further exists a need to quickly capture a large part (or all) of the liquid that is present in the breath for detection and analysis.

### SUMMARY OF THE INVENTION

Broadly, the present invention provides methods, devices and kits for collecting and analyzing a biological sample, e.g., in the form of liquid particles, aerosol particles and/or vapor by capturing them in a device comprising a surface and chamber space that condenses or freezes the biological sample when a user exhales one or more breaths through the device. Capture can be performed on the freezing surface immobilizing water upon contact. The chamber space within the device may freeze liquid breath particles and vapor to collect them. The collected sample may be a frozen sample, a combination of liquid and frozen sample or liquid sample, for example depending on the temperature of a capture surface and the time between collection and processing. After collection, the liquid biological sample is gathered and collected for example, by draining, pushing, scraping or centrifugal force into a vial or be taken up to transfer with a pipette. The liquid may be collected and combined with a sample preparation reagent such as a virus lysing reagent, an internal standard, etc. Sample may be collected remotely and mailed or may be collected at the point of care. One sample may be collected, or several samples may be collected and processed in parallel. After collection, the sample is segmented and analyzed. Analysis may be performed by PCR, qPCR RT-PCR, RT-qPCR, digital PCR, LAMP or any nucleic acid detection method, or by antigen, lateral antigen, protein, ELIZA, nucleic acid transducer, protein transducer, mass spectrometry, spectrophotometry or any analytical tool or method. Nucleic acid amplification reagents or associated reagents may contain a lysing reagent such as acetonitrile.

At its most general, the present invention provides a method for analyzing a breath condensate sample by separating, digitizing or segmenting the sample into independently analyzable units to enable the number and rate of pathogens shed to be quantified. Each independently analyzable unit in which a positive detection occurs represents a pathogen unit, molecule or cell. Segmented detection provides a method for quantifying an amount of pathogen being shed by an individual and the degree of an individual's infectiousness.

In a first aspect, there is provided a method of determining a quantity of a target in a biological sample, the method comprising: obtaining a biological sample by collecting breath condensate over a sample collection period; segmenting the biological sample into a plurality of independently analyzable units; analyzing the segmented biological sample to determine a number of units amongst the plurality of independently analyzable units having at least one copy of the target present therein; and determining the quantity of the target in the biological sample based on the determined number of units, wherein the target comprises a nucleic acid, and wherein analyzing the segmented biological sample comprises performing isothermal amplification of the nucleic acid in each of the plurality of independently analyzable units. This aspect of the invention provides a rapid technique for pathogen quantification that does not rely on the use of thermocycling equipment that is needed for known techniques such as qPCR. Isothermal amplification and detection may be performed by LAMP, RPA or any other established detection method.

In a second aspect, there is provided a method of determining a quantity of a target in a biological sample, the method comprising: obtaining a biological sample by collecting breath condensate over a sample collection period; segmenting the biological sample into a plurality of independently analyzable units; analyzing the segmented biological sample to determine a number of units amongst the plurality of independently analyzable units having at least one copy of the target present therein; and determining the quantity of the target in the biological sample based on the determined number of units, wherein the sample collection period is less than 5 minutes. The segmenting or digitizing technique disclosed herein is capable of determining quantitative information about the amount and shed rate of the target from a relatively small sample size. This means that the period in which breath is collected can be set at a practical level. The period may be further reduced for example when the method makes use of one of the efficient breath condensate collection techniques discussed below.

In a third aspect, there is provided a method of determining a quantity of a target in a biological sample, the method comprising: obtaining a biological sample by collecting breath condensate over a sample collection period; segmenting the biological sample into a plurality of independently analyzable units; analyzing the segmented biological sample to determine a number of units amongst the plurality of independently analyzable units having at least one copy of the target present therein; and determining the quantity of the target in the biological sample based on the determined number of units, wherein a volume of the biological sample that is segmented into the plurality of independently analyzable units is equal to or less than 500 μL. As mentioned above, the segmenting or digitizing technique disclosed herein is capable of determining quantitative information about the amount and shed rate of the target from a relatively small sample size. The volume of the biological sample that is segmented may be the volume collected from the breath condensate, e.g. before mixing a solvent or the like.

Features of the aspects above may be combined in any manner. These aspects may include any one or more of the following features.

The method may further comprise determining a shed rate based on (i) the determined quantity of the target in the biological sample and (ii) a duration of the sample collection period. For example, the quantity may be divided by the duration to obtain a shed rate whose units are number of pathogens per unit time, e.g. per minute.

The method may further comprise outputting a report of the quantity of the target in the biological sample. The output report may comprise a quantitative or qualitative indication of a shed rate of pathogen in the biological sample.

For example, the method may further comprise comparing the determined shed rate to a threshold, and generating a notification if the determined shed rate exceeds the threshold. For example, the threshold may correspond to a predetermined infection risk level. In this scenario, the notification may comprise a warning that the user from whom the biological sample was obtained is infectious.

In another example, the output report may indicate if the target is present in an amount above or below a detection limit.

Determining the quantity of the target in the biological sample may use the equation

Nλ=In(N/N−x)
where N is the number of independently analyzable units, x is the determined number of units, and Nλ is the number of copies of the target across all of the plurality of independently analyzable units.

Alternatively or additionally, determining the quantity of the target in the biological sample may comprise: determining a maximum quantifiable threshold based on total number of independently analyzable units; and, if the determined number of units is the total number of independently analyzable units, determining that the quantity of the target in the biological sample is equal to or greater than the maximum quantifiable threshold. These features recognize that quantification is not possible if the maximum quantifiable threshold is exceeded, and therefore in such a scenario the method may output a report that indicates that the detected amount of the target has exceed the maximum quantifiable threshold.

Segmenting the biological sample into a plurality of independently analyzable units may comprise dividing the biological sample between less than 5000 independently analyzable units, for example by separating the biological between at least 2 but not more than 1000 independently analyzable units.

The biological sample may comprise nucleic acids. Analyzing the segmented biological sample may comprise detecting nucleic acid amplification at each of the independently analyzable units. As discussed above, in one aspect of the invention isothermal amplification is performed. However, in other aspects different amplification techniques may be used, e.g. thermocycling techniques such as PCR. For example, the segmented biological sample may be analyzed by any of LAMP, RPA, RT-PCR, LC, LC-MS, GC, GC-MS, Lateral Antigen.

Segmenting the biological sample may comprise depositing each portion of a plurality of portions of the biological sample into a respective well amongst a plurality of wells, each well being a respective one of the plurality of independently analyzable units. Each well may be a physically distinct sub-volume from which an independent detection result can be obtained. The plurality of wells may form an array suitable for receiving the biological sample, e.g. from a collection chamber in which it is obtained. Segmenting the biological sample may comprise depositing an equal volume of the biological sample into each well. In other words, each portion of the biological sample has the same volume. That volume may be 5 μL or less. Each well may have a size commensurate with the volume it is to receive. For example, each well may be configured to retain a liquid volume equal to 5 μL or less.

The method may include mixing the biological sample with one or more detection reagents before analysis. The mixing may occur in the collection chamber (e.g. before segmenting) or may occur in the plurality of analyzable units (e.g. after segmenting). For example, each of the analyzable units may have an organic solvent stored therein. The quantity of organic solvent stored in each analyzable unit may be 5% or more of the volume of the respective portion of the biological sample that is to be received in the analyzable unit. The organic solvent may be a water miscible solvent and/or an aprotic solvent.

In some examples, all of the collected biological sample may be segmented into the plurality of independently analyzable units. Preferably at least half of the biological sample is segmented into the plurality of independently analyzable units. This may be done for example to ensure that the same volume of breath condensate is processed for different users.

The duration of the sample collection period may be between 10 seconds and 5 minutes. For example, the duration of the sample collection period may be 2 minutes or less, 1 minute or less, 30 seconds or less, or 20 seconds or less, or the duration of the sample collection period may be 20 seconds or more, 30 seconds or more, 1 minute or more, or 2 minutes or more. Preferably the duration of the sample collection period is approximately 20 seconds, or between 20 and 30 seconds.

Obtaining the biological sample may include capturing six or fewer exhaled breaths of a user within the sample collection period. The method may include recording the sample collection period.

Obtaining the biological sample may comprise receiving breath exhaled by a user in a breath collection device, and cooling the exhaled breath to form the breath condensate containing the biological sample. These steps may utilize any of the breath condensate collection devices disclosed herein. For example, cooling the exhaled breath may include contacting the exhaled breath with a capture surface, the capture surface being cooled by a coolant to cool the exhaled breath.

The method may use a device for collecting and cooling a breath sample using an endothermic coolant. This may permit the entire collection and cooling apparatus to be disposable, which may for example allow collection to occur in a wide variety of environments, rather than only in places having specific cooling capability.

Such a device for collecting a biological sample from breath of a user may comprise: a tube adapted to allow the user to exhale their breath into the device; a collection chamber in fluid communication with the tube, the collection chamber having a capture surface; a receptacle for receiving the collection chamber, the receptacle containing a coolant that undergoes (i.e. is arranged to support or perform) an endothermic process to cool the capture surface to a temperature below the freezing point of water, whereby the biological sample from the breath of the user condenses or freezes on the capture surface of the collection chamber.

In this example, the coolant acts to cool the breath condensate using an endothermic cooling process. An endothermic process is any thermodynamic process with an increase in the enthalpy H (or internal energy U) of the system. In such a process, a (closed) system absorbs thermal energy from its surroundings.

An endothermic process may be a chemical process, such as dissolving ammonium nitrate in water, or a physical process, such as melting e.g. ice melting, vaporization and sublimation. Endothermic processes may be a combination of chemical and physical processes such as adding sodium chloride or magnesium chloride added to ice and melting, i.e. salt ice.

The device may be cooled by salt ice, dry ice or a combination of cooling materials or by cooling chemicals. The cooling materials may be in direct contact with the outside of the collector chamber surface.

In a further aspect, the present invention may provide a method for detecting a target in a biological sample from breath from a user, wherein the method comprises:
(a) directing at least one breath of at least 10 seconds from a subject into a breath collection device, wherein the breath collection devices comprise a collection chamber in thermal communication with a coolant undergoing an endothermic process, wherein the collection chamber is capable of capturing at least a portion of the at least one breath as frost, ice or liquid to form a captured volume;
(b) processing the captured volume to recover one or more components of the biological sample;
(c) segmenting the one or more components into a plurality of independently analyzable units; and
(d) analyzing the segmented one or more components to detect the presence of the target, thereby detecting a target in a biological sample from the breath from a subject.

The method may optionally include allowing a frozen biological sample to melt to form a liquid biological sample for analysis, and/or processing the frozen or liquid biological sample e.g. before analysis.

The device may comprise a turbulence inducer disposed in or around the tube to cause the flow of breath to become turbulent to enhance contact between the capture surface and the exhaled breath of the user.

In some cases, the tube has a first end for the user to exhale into the device and the collection chamber is a vial having an interior capture surface, the vial being disposed over a second end of the tube, wherein the flow of breath reverses around interior walls of the vial so that the biological sample condenses or freezes on the capture surface. The collection chamber may be an end of the tube or the tube may incorporate a vial, e.g. a removable vial for facilitating processing of the collected sample.

The turbulence inducer may be a separate component to the tube or collection chamber, for example an insert, or may be provided by the tube or collection chamber having structures, e.g. a rough surface or protrusions, that affect the flow of breath passing over them to induce turbulent flow.

As explained further herein, in some instances, the collection chamber is a syringe barrel, the tube fits into the barrel of the syringe and the turbulence inducer fits around an outer surface of the tube.

The collection chamber may terminate in a closed end collector, such as a closed end tube or vial. For example, the syringe barrel may be a closed end tube or may terminate in a vial, cap or needle.

In some cases, the tube is open at a first end to allow the user to breathe into the device and comprises a wall towards a second end to deflect the breath of the user over the capture surface to enhance contact between the capture surface and the breath of the user.

Additionally, or alternatively the vial and/or the tube are removable to facilitate processing of the biological sample or to provide a multi-use device through replacement of the vial and/or tube.

The capture surface may comprise or consist of an inside wall of the collection chamber. In some embodiments breath condensation may occur solely on an inside wall of the collection chamber, i.e. may not occur within the tube or on the turbulence inducer. In some embodiments liquid breath condensate coalesces and is collected from the inside wall of the collection chamber.

The collection chamber may have a capture surface having a surface area equal to or less than 75 cm², or equal to or less than 50 cm². The collection chamber may have a volume between 0.5 and 50 mL.

The coolant may be capable of cooling the capture surface to a temperature between about −10° C. (optionally about −20° C.) and about −30° C., −40° C., or −70° C. The biological sample from the breath of the user may condense or freeze on the capture surface of the collection chamber within about 10 to 120 seconds to provide a biological sample having a volume of between about 20 μL and 250 μL, optionally 180 μL.

The coolant may be any suitable substance that undergoes an endothermic process capable of imparting a cooling effect suitable to condense or freeze the collected breath on the capture surface. The coolant is preferably a disposable substance. For example, the coolant may be dry ice. In other examples, the coolant may be a coolant mixture comprising two or more components. The components may include water, e.g. ice, and a salt or other substance which when mixed with ice lowers the melting point of the ice. For example, the components of the coolant mixture may comprise any of:
a) water and ice;
b) water and ammonium chloride;
c) water and sodium nitrate (III);
d) water and ammonium thiocyanate;
e) ice and sodium acetate;
f) ice and ammonium chloride;
g) ice and sodium chloride;
h) ice and ammonium nitrate (V);
i) ice and sodium bromide;
j) ice and potassium chloride;
k) ice and magnesium chloride;
I) ice and sulfuric acid (VI) 66%; and
m) ice and calcium chloride hexahydrate.

The device may comprise a mixing vessel for combining and mixing the components of the coolant mixture. In this way, the endothermic process may be initiated at the point of use of the device. This may be advantageous because it optimizes (e.g. maximizes) the cooling effect on the collection chamber. The mixing vessel may comprise a first mixing container and a second mixing container. The first and second mixing containers may be connectable in a first configuration to define an enclosed mixing volume in which the components of the coolant mixture are to be combined and mixed. For example, the first and second mixing containers may be open containers whose mouths can be interconnected to form a connected assembly.

In a particularly advantageous arrangement, one or both of the first and second mixing containers may also provide the receptacle. For example, the first and second mixing containers may be connectable in a second configuration to define the receptacle. In one example, the first and second mixing containers may each comprise a tubular section, and the first mixing container may be dimensioned to be sleeved around the second mixing container in the second configuration. In this configuration, the second mixing container may contain the coolant mixture and the first mixing container may act as a thermal insulator.

In a further aspect, the present invention provides the use of a device as defined herein for collecting a frozen or condensed biological sample from breath of a user to detect a target in a biological sample by segmenting the biological sample.

In a further aspect, the present invention provides a kit comprising a device as described herein, wherein the kits comprises a plurality of disposable elements of the device and/or reagents for processing the biological sample. The disposable elements may comprise any one or more of the collection tube, the receptacle, the turbulence inducer, the collection chamber, the coolant (or one or more components of the coolant) and optionally a plastic mouthpiece cover.

The coolant may be disposable. The coolant may be salt ice, coolant chemical mixture and/or dry ice endothermic process. The coolant may be in direct physical contact with a disposable collection chamber.

The collection chamber may be disposable. The collection chamber may be a syringe barrel with a breath introduction tube inserted. The syringe barrel may have a vial, needle or cap attached.

The receptacle may be disposable. The receptacle may be a single use item. For example, the receptable may be made from paper and/or plastic material. The receptable may be substantially devoid of metal. In one example, the receptacle may be a pair of nested paper cups.

The receptable may comprise a retainer to hold the collection chamber in contact with the coolant. For example, the receptacle may comprise a lid for covering a volume in which the coolant is held. The lid may include a clip, aperture or other means for receiving and retaining the collection chamber.

In other examples, the breath condensate may be collected on a cold surface that has been super cooled, e.g. by using a Peltier cooler, a circulating cooler containing liquid below water freezing temperature, circulating evaporation cooler, a device cooling from a device releasing gas such as compressed carbon dioxide, a device that contains or has been treated with liquid nitrogen or dry ice.

The phrase “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

All of the references mentioned herein are expressly incorporated by reference in their entirety.

The present invention will now be described by way of examples and not limitation with reference to the accompanying examples and figures.

### BRIEF DESCRIPTION OF THE FIGURES

FIG. 1 is a schematic diagram of a breath collection device that can be used in embodiments of the present invention.

FIG. 2 depicts an enlarged portion of the device shown in FIG. 1 showing how the breath flows through the end of the straw and into the vial, with the biological sample captured as liquid and/or ice on the inside of the vial.

FIG. 3 is a schematic diagram of another breath collection device that can be used in embodiments of the present invention.

FIG. 4 shows the breath collection device of FIG. 3 with a horizontal breath inlet.

FIGS. 5A and 5B show a plunger that equalizes the total volume of collected liquid by redirecting any excess liquid.

FIGS. 6A-6D illustrate the components, assembly and use of a collection device that can be used in embodiments of the invention.

FIG. 7 depicts a collection apparatus that uses the breath collection device of FIG. 1 with an active cooling system.

FIG. 8 depicts a collection apparatus that uses the breath collection device of FIG. 4 with an active cooling system.

FIG. 9 shows the collection of liquid from the tube vial of the invention compared to a conventional tube.

FIG. 10 shows a flow diagram of the sample segmenting process used in determining the amount of virus or pathogen shed and the shed rate of virus or pathogen from an individual.

### DETAILED DESCRIPTION OF THE INVENTION

Humans exhale approximately 200 μL of aerosol liquid (mostly water) every minute. A rapid sampling of breath condensate may limit the sample volume collected thus limiting the sample volume processed for detection. A pathogen detection method based on 1-5 μL sample liquid volume represents only a few seconds of exhaled breath and because pathogens can be at low concentration in exhaled breath the sample volume may be too small and may not contain any pathogen.

In addition, if only a fraction of the liquid contained in the total exhaled breath is captured, then it is possible that only a fraction of an individual's shed and exhaled pathogens have been captured thus increasing the size of the sample needed to be collected.

Water vapor and water aerosol particulates are present in breath and can be deposited as liquid and/or ice/frost on a tube or vial wall when the temperature of the tube is significantly below the freezing point of water. In the devices of the present invention, tube or vial wall includes a collection surface capable of being kept at a temperature that is typically in the range of the −10° C., −15° C., −20° C. to −40° C. or colder. This means that water vapor, particulates and/or aerosol particles in the breath are deposited on a capture surface of a collection chamber where they can become solid and form ice crystals in the devices of the present invention or else condense on the cold capture surface, e.g. as droplets of liquid.

Advantageously, in order to collect breath condensate quickly and effectively, the freezing surface must be accessible to exhaled air, e.g., by arranging the collection chamber so that it is in fluid communication with a tube or straw through which the user of the device can exhale. Preferably, the device is adapted so that the capture surface can be maintained at the supercooled temperature to minimize the phenomenon that as frost is collected on the capture surface of the collection chamber, the temperature of the frost at the surface rises, potentially inhibiting the collection of further moisture and possible leading to inconsistent collection. It may also be advantageous to avoid the capture surface coming into contact with ambient air before collection takes place, to prevent a portion of the collected sample of frost to come from the ambient air, rather than from the breath of the user. To this end, in some cases, preferably the freezing capture surface is shielded from ambient air until the device sample is introduced to the collection surface so that the capture surface is protected from contact with air other than in the breath exhaled by the user. For example, in the devices of the present invention, a tube or straw is insertable into the device and into the collection vial past a barrier or shield to allow the delivery of the breath sample to the freezing capture surface. Prior to insertion of the tube or straw, the capture surface is effectively shielded from ambient air until the breath sample is introduced into the device. In one embodiment, the capture surface is a tubular vial with a means of collecting the liquid from the sample when removed from the freezing source, for example enabling the collection and processing of a sample having a volume of 200 μL or less, or having a volume of 250 μL or less.

In addition, in the prior art, it is very difficult to collect very small sample volumes with normal breathing apparatus sampling, with the result that large sample volumes must be collected over many minutes to enable downstream processing. The devices and methods of the present invention are capable of rates of sample collection of up to 2 μL/s, and more preferably up to 3-4 μL/s. After collection, the biological samples can be recovered as liquid and subjected to subsequent processing.

The collection structure of the devices of the present invention can take the form of a vial or a tube connected to a vial at the end of the tube. The terms “vial” and “tube vial” are used interchangeably in the present specification. In some embodiments of the invention the collection structure of the device is a tube. Alternatively, the collection structure of the device is a syringe barrel with the plunger barrel removed and the syringe bottom capped or sealed. The tube and/or vial of the apparatus is a supercooled surface that is a flat or curved, etc., smooth or rough surface and may contain grooves and depressions to facilitate collecting liquid. The outside of the tube and/or vial is cooled while breath is introduced inside with a breathing insert tube. In some embodiments of the invention the breathing insert is a (disposable) straw. Sample is directed into a tube and collected into the bottom of a vial. The freezing surface of the vial is protected from ambient air until breath is introduced. The shield is removed, and frozen breath condensate is collected. The breath enters the device in a laminar flow from the mouth. In some embodiments of the device the laminar flow is disrupted to produce turbulent flow as the breath flows across the cold surface. The introduction of turbulence may be performed as the breath reverses flow at the distal end of the collection tube vial. In some cases, the collection structure is a vial, e.g., a removable vial or the tube incorporates a collection vial.

The collection structure or collection chamber of the device of the present invention can take the form of a syringe barrel with a breath introduction tube and having turbulence inducer inserted inside the syringe barrel. The syringe barrel can have a vial and/or a needle connected to the end. This can close the end of the syringe barrel. After collection of the breath condensate, the syringe barrel may use the inserted tube as a plunger or a new plunger may be inserted to coalesce and collect the condensed liquid. Liquid may be expelled and drawn up by the syringe via a needle. The end of the syringe may have a luer fitting or luer lock fitting or another fitting to attach the needle.

The end of the collection device may be blocked, and air does not pass through the vial, tube or syringe. Because the end is blocked, the breath capture device reverses or changes flow through device. Reverse breath flow reduces the diffusion distance to the cold surface. The air flow may be laminar or turbulent. Breath liquid from particles and vapor are deposited or drained directly into a vial drain or directly into a vial. The vial containing captured liquid may be used directly for processing and detection including nucleic acid detection. The vial may contain lysing solvent. The vial may contain amplification reagents.

The present invention described collects sufficient breath for analysis and virus detection with as little as 10 seconds up to 2 minutes of breath.

Materials collected in the liquid and frozen condensate could potentially include virus, bacteria, nucleic acids, organic compounds, volatile inorganic compounds, proteins, or biological materials, present in the breath. Those materials that are present in the breath will be collected by the device and method of the invention.

After collection, the sample is analyzed. Analysis may be performed on the collected materials to detect nucleic acids, utilizing devices that amplify and/or tag and then detect and optionally quantify. Other detection devices and methods include mass spectrometry, LC/MS, spectroscopy, UV and VIS spectroscopy, IR spectroscopy, gas chromatography, liquid chromatography, sequencing, next generation sequencing, culturing, colony counting, isothermal and thermocycling nucleic amplification, tagged and direct, hybridization, CRISPR, respiratory panel, etc. Applications of the technology include detection of viral and bacterial infections that spread by breath, as well as other disease states based on chemicals exhaled. Crucially, if infectious agents are being exhaled or coming out a person's mouth, by definition, the person is infectious. Virus or bacteria becomes airborne or spatters and can infect another person. The device may therefore be used as a tool for research and/or diagnostics.

The breath intake or sample inlet structure of the device of the invention may be vertical, horizontal or in between vertical and horizontal. A vertical breath intake may be positioned straight down into the device while horizontal is 90 degrees to the instrument. The collection tube to which the intake is connected may be positioned in any orientation. In some embodiments of the device, a horizontal or partly horizontal sample inlet may be employed. Partly horizontal shall mean the breath introduction is within 45 degrees of horizontal. In some embodiments of the device, a vertical sample inlet may be employed. This device will capture breath, gaseous water and liquid particulates. A horizontal mode allows the capture of breath without the capture of spit or dribble. In addition to being horizontal, the breath intake may include a depression or trap to capture spit or very large liquid particles.

In some embodiments of the device, a vertical sample inlet may be employed. Vertical intake means breath from the mouth is located directly above the apparatus and breath is directed down into the apparatus. Partly vertical shall mean the tube structure is within 45 degrees of vertical. A vertical or partly vertical breath entry orientation can be advantageous. In addition to capturing gaseous water and small airborne particulates, larger liquid particles, spit or droplets may also be captured. Some people emit respiratory spray as they breath, talk or sing. This can vary from person to person and with some people producing very large droplets while other people producing quite a lot of large droplets while breathing, talking or singing. In some embodiments of the device, the breath inlet mouthpiece may be constructed to capture breath exhaled and when speaking or singing. In some embodiments of the invention, the mouthpiece is constructed to cover a portion of the lips to facilitate sampling by a combination of breathing, talking and/or singing.

A vertical or partly vertical capture breath inlet directs breath gas/liquid, small breath particulate and large breath particulate including airborne and spit particulate.

A vertical or near vertical breath intake capture device can capture breath gas/liquid, small breath particulates and large breath particulates, spit and dribble, thus measuring the potential infectiousness of different modes of disease expulsion from an individual while a horizontal or near horizontal breath intake capture device will limit capture to breath gases and liquid particles large enough to remain in the breath.

Regardless of how liquid particulates are introduced into the air or what type and how they are captured, the device of the invention may be used as a tool for research and/or diagnostics. For example, the viral infectiousness of a particular person depends not only on the ability of the virus to infect but is also a measure of the person to release and transport virus to another individual. Public health safety is affected more by the presence of infectious individuals in a crowd than by the presence of infected individuals in a crowd. The capture and collection of the water vapor and particles is efficient and effective. The collection is easy, meaning the procedure is quickly performed with minimal effort and no discomfort to persons providing breath samples.

In addition to liquid collection from breath, the sample liquid may be collected from ambient spaces. Air may be pumped through the device to collect and detect materials that may be present in the ambient air of a room or building or even outside a building.

In some embodiments of the invention, there is provided a method of rapidly quantifying breath pathogen shed, e.g. by determining an absolute number of shed pathogen in the collected sample and/or a pathogen shed rate. The method may be characterized by capturing all, most or a defined percentage of exhaled pathogens. In addition, the collection devices discussed herein may enable a large breath condensate sample to be captured, which in turn permits detection of pathogens shed at a low rate.

In some embodiments of the invention, the collection breath condensate sample has a volume equal to or larger than any of 10, 15, 20, 30, 40, 50, 60, 75, 100, 125, 150, 175, 200, 25 and 300 μL. The sample is processed to measure the total pathogens contained therein. For example a sample having a volume in the range 20-200 μL can be collected and processed. In some embodiments of the invention, the sample collection period (i.e. the duration in which breath is collected) may be prescribed. For example, the breath sample may be collected for a duration equal to or less than any of 5 min, 2 min, 1 min, 30 sec, 20 sec, 15, sec, 10 sec and 5 sec. In some embodiments of the invention, a defined amount or known fraction of the collected sample is processed for detection. In some embodiments of the invention, at least 50% of the sample is processed for detection.

In embodiments of the invention discussed herein, the sample is segmented. The number of segments into which the sample is divided may be tuned to enable a low level of virus in the sample to be detected and a corresponding shed rate to be reported. In some embodiments of the invention, the breath condensate sample is segmented into two or more digits. For example, the breath condensate sample may be segmented into 3, 4, 5, 6, 8, 10, 15, 20, 25, 30, 40, 50, 60, 70, 80, 90, 100, 200, 300, 400, 500, 600, 700, 800, 900, 1000, 2000, 3000, 4000, 5000 or more digits. A relatively large sample volume may be needed to capture and detect a low level of virus or pathogen.

### Definitions

Efficient capture or collection means that a large part or all the water vapor and liquid present in the breath sample is captured.

Effective capture or collection means that the collection procedure can be done rapidly with collection and preparation of the sample for processing the sample in less than 10 minutes, less than 5 minutes, less than 2 minutes or less than 1 minute.

Ease of collection means that the procedure is quickly performed with minimal effort and no discomfort to persons providing breath samples.

The collection vial in the apparatus of the invention is any type of closed tube or structure where liquid can be collected directly from the collected sample. A tubular vial of the invention has a means to collect the liquid from the sample. Any method including gravity, scraping, momentum or centrifugal force may be used to coalesce and collect the liquid from the breath into the vial.

The collection tube of the apparatus/device of the present invention may be any type of tube or structure where liquid can be collected directly from the collected sample. In some cases, the collection tube may be a closed end tube or end in a vial. For example, the collection device may comprise a syringe barrel that terminates in a closed end vial.

Frost or frozen breath is defined as any water vapor or water aerosols that is collected from exhaled breath in the device and method of the invention. The water collected can be primarily or partially a solid, but also some portion may be in the form of liquid or may melt quickly when the device is removed from the cold source or as sample collection proceeds and the device warms.

Super cold temperature may be defined as being −10° C. or lower or being cold enough to capture at least some portion of the breath vapor or breath liquid particles as ice or frost, i.e., providing a frozen or partially frozen sample. Super cold temperatures can range from about −10° C. to −40° C. or even −70° C. or colder.

The collection vial is optional and defined as a chamber or vial where liquid from a breath sample can be directed for collection, additional processing or storage. A needle or cap may be positioned on the collection device in place of a collection vial or chamber.

The term “pathogen” is used herein to mean bacteria, virus, fungi, protozoa, worms or other microorganisms that can cause disease.

The term “shedding” is used herein to refer to the act of emitting pathogens (e.g. viruses, bacteria, etc. as defined above) in exhaled breath condensate. In this context, breath condensate includes respiratory spray that may be caused by drooling or excess saliva. Shedding may be quantified by a number or pathogens or a rate at which pathogens are shed. For example, shedding may be characterised by a quantitative number of pathogens shed, i.e. an absolute number of pathogens detected for a given sample. In practice, such a quantitative number may be corrected based on detection sensitivity.

The term “shed rate” is used herein to mean a rate at which pathogens are emitted per unit time. Aspects of the invention may be configured to determine a quantitative shed rate based on a number of virus detected within a given or known sampling time, where the quantitative shed rate is expressed as number of pathogen shed per minute (or other suitable time unit). Alternatively or additionally, aspects of the invention may be configured to determine a qualitative shed rate, which is shed rate defined qualitatively e.g., zero, low, medium, high and very high. Other qualitative terms may be used.

Aspects of the invention discussed herein discuss the process of “digitizing” or “segmenting” a breath sample. These terms may be used synonymously to refer to the process of splitting a collected sample into two or more separate sample volumes, which are independently analyzable units. In some embodiments, droplets may be formed or multiple sample wells may be filled to divide sample into discrete volumes.

The independently analyzable units may each be referred to as a “segmented breath sample volume” or in some cases a segmented “digit”. For example, in aspects of the invention a volume of collected breath condensate is separated into digits. The segmented digits may include intact virus or pathogen or lysed nucleic acid. The segmented digits may include detection reagents. In some examples, a portion of the sample volume may be segmented. For example, it may be desirable to segment the same volume across a plurality of samples taken from the same user or different users. The sample volume to be segmented may be in the range 1-1000 L, 5-500 μL, 10-400 μL, 20-300 μL, 30-200 μL, 40-150 μL, or 50-100 μL.

In some examples of the collection apparatus discussed herein, cooling of the tube or vial collecting frost may be accomplished by inserting the collection structure into a coolant in which an endothermic process is underway. Endothermic processes are defined as processes where reactant material absorbs heat energy from the surroundings. The endothermic process draws heat into the coolant from the surrounding environment and hence causes a cooling effect on the collection structure, e.g. so that an inner surface of the collection device presents a super cooled surface to the incoming breath. The endothermic process may be a chemical or physical process or a combination of chemical and physical processes. Examples of such endothermic processes include evaporating liquids, melting ice, dry ice sublimation, liquid expanding into gas, salt dissolving in water and combinations of these processes. The coolant may be a coolant mixture comprising two or more components that undergo an endothermic process when combined.

A chemical reaction or physical change is endothermic if heat is absorbed by the system from the surroundings. In the course of an endothermic process, the system gains heat from the surroundings and so the temperature of the surroundings decreases. Endothermic reactions are chemical reactions in which the reactants absorb heat energy from the surroundings to form products. These reactions lower the temperature of their surrounding area, thereby creating a cooling effect. Physical processes can be endothermic as well. For example ice cubes absorb heat energy from their surroundings and melt to form liquid water.

Some examples of endothermic processes include:
The reaction of barium hydroxide octahydrate crystals with dry ammonium chloride
Dissolving ammonium chloride in water
The reaction of thionyl chloride (SOCl2) with cobalt(II) sulfate heptahydrate
Mixing water and ammonium nitrate
Mixing water with potassium chloride
Reacting ethanoic acid with sodium carbonate
   Ice or ice water may replace liquid water. Some examples of endothermic processes
Melting ice or salt ice
Evaporating liquid water, ammonia or carbon dioxide.
Liquid carbon dioxide expanding to gas
Separating ion pairs

Cooling of the tube or vial collecting frost may also be accomplished using a number of different strategies, which may be used in addition to or as an alternative to the endothermic coolant discussed above. These include but are not limited to a cold surface that has been super cooled including using a Peltier cooler, a circulating cooler containing liquid below water freezing temperature, circulating evaporation cooler, a device cooling from a device releasing gas such as compressed carbon dioxide, a device that contains or has been treated with liquid nitrogen or dry ice and other methods.

Although invisible to our eyes, water vapor and water particulates are always present in breath. The dew point is the temperature when liquid will form condensate from breath. Frost will be collected when the temperature is below the dew point and below the freezing point. Breath frost is water vapor and particulates that become solid and form ice crystals in the device of the invention or condenses on the cold capture surface. In the device and method of the invention frost or liquid is formed and collected from water and from air that is at ambient or body temperature when introduced into the device.

The device and method of the present invention capture water liquid from aerosol particles and vapor easily, efficiently and effectively from a breath sample for viral, bacterial, biological and chemical analysis. Efficient capture means that a large part (or all) of the water liquid present in the breath sample is captured and available to be processed for detection. It is important to collect all of the breath sample liquid in which virus, bacteria or chemicals may be present. If only the easiest collectible portion of the sample is collected, e.g., large liquid particles, then it is possible that a non-representative sample was collected.

As the present invention uses devices with super cold surface temperatures, collection is generally more efficient at the beginning of collection process and collection efficiency decreases as the volume of breath collected increases and sample is collected. The surface ice, frost or liquid formed will decrease the efficiency of collection because the temperature of the surface is warmed and can't be cooled as much or as quickly. In addition, as the devices of the present invention are generally small, this allows the water that is captured by the device to be more easily coalesced and collected for processing. This works against capturing liquid since the mass of the collection device is small because the device is small. As the device size decreases, the amount of liquid that can be collected also decreases. Capturing all or most of the water liquid in the breath may be efficient only for the first 10, 15, 20, 25 or 30 seconds or for the first 1, 2 or 3 minutes and then will decrease. However, by this time sufficient breath liquid and vapor is collected for detection of the desired material. All the captured liquid may be processed for detection. In some cases, the methods of the present invention comprise a further step of processing all or a portion of the captured liquid biological sample, for example to enable the detection of a target present in the sample. In some cases, at least 25% of the captured liquid sample is used for downstream processing. In some embodiments, at least 50%, 75%, 80%, 85%, 90%, 95% or an even greater percentage of the sample is processed, for example to improve the sensitivity of detection of the target present in the sample.

The capture or collection of the water vapor and particles is efficient in the device and method of the invention meaning that a large part or all the water vapor and liquid present in the breath sample is captured. The capture or collection of the water vapor and aerosol particles is effective meaning the collection procedure can be done rapidly. Collection of the sample may be performed in less than 5 minutes, less than 4 minutes, less than 3 minutes, less than 2 minutes, less than 1 minute, less than 45 seconds, less than 30 seconds, less than 20 seconds, less than 15 seconds, less than 10 seconds or less than 5 seconds. Collection and preparation of the sample for processing can be performed in less than 10 minutes, less than 5 minutes or less than 2 minutes. Collection and preparation of the sample for processing can be performed between 5 seconds and 5 minutes, between 15 seconds and 45 seconds, between 10 seconds and 4 minutes, between 15 seconds and 3 minutes, between 20 seconds and 2 minutes, between 25 seconds and 1 minutes. Collection can be performed over at least 5, 10, 15, 20, 30, 50, or 60 seconds. Effective means that the collection and procedure can be done rapidly and the detection process may be initiated and started quickly after the start of sample collection, often in just a few minutes. The detection process may be initiated in less than 10 minutes or less than 5 minutes. This includes lysing of the sample with an organic solvent. PCR detection or LAMP detection which can be performed as quickly as 20 minutes; however, this technology is advancing rapidly, and detection times are likely to decrease further.

The cold surface area of the device of the invention is small because of the desire to capture and process small amounts of liquid. In some embodiments of the invention, the vial volume that liquid is collected into is 5 mL, 4 mL, 3 mL, 2 mL, 1 mL, 0.5 mL, 0.2 mL, 0.1 mL, 0.05 mL or less. In some embodiments of the invention, the cold surface area that ice forms on is 100 cm², 75 cm², 50 cm², 40 cm², 30 cm², 20 cm², 10 cm² or less. Although the temperature of the collection surface may increase as sample is collected, in some embodiments of the invention the initial temperature of the cold surface is below 0° C. In some embodiments, the initial temperature of the cold surface can be −10° C., −15° C., −20° C., −25° C., −30° C., −35° C., −40° C., −45° C., −50° C., −55° C., −60° C., −65° C., −70° C., −80° C. or colder. The capture surface of the collection chamber may remain at a temperature between 0° C. and −80° C., between −15° C. and −70° C., or between −20° C. and −40° C. during the collection process.

To collect breath frost quickly and effectively, the freezing surface must be easily accessible. However, if a freezing surface is exposed, frost and liquid could inadvertently be collected from any ambient air. To prevent this, the surface may be shielded until the sample is introduced to the surface, for example with a barrier. However, once collection of ice starts, the collected ice on the surface raises the collection temperature, which lowers the efficiency of further collection. Further collection is possible, but collection may occur at a slower rate if the cold interior surface temperature cannot be maintained and is raised.

In addition, sampling very small volumes of liquid available from breath is difficult with normal breathing apparatus sampling. Frost formation on surfaces will prevent collection of further frost. In the device of the present invention, very small volumes of liquid are collected and manipulated. The volumes of liquid collected can be less than 500 μL, less than 400 μL, less than 300 μL, less than 200 μL, less than 100 μL, less than 80 μL, less than 50 μL, less than 40 μL, less than 30 μL, less than 25 μL, less than 20 μL, less than 15 μL, less than 10 μL, less than 5 μL, or in the range of 5-100, 10-100, 15-300 or 20-100 μL. In the device and method of the invention, sufficient liquid can be collected from less than 10 exhaling breaths, less than 9, 8, 7, 6, 5, 4, 3, or less than 2 exhaling breaths. In the device and method of the invention, usable liquid can be collected from even 1 exhaled breath. Usable liquid from one adult's exhaled breath may be more than 100 μL and a significant portion, generally in the range of 20-80 μL, can be captured.

To achieve capture of these sample volume, the volume of the collection chambers of the devices of the present invention is generally smaller than those used in prior art devices that collect breath samples. The collection tube of the invention inlet diameter and tube length will have an effect on the collection capacity and resistance to breath inlet flow. In some embodiments, the tube size may be based on 1, 3, 5, 10 or 20 mL syringes or even larger syringe barrel volumes. As the syringe volume increases, commercial syringe barrel bodies have larger diameters. This can allow the breath inlet tube diameter to increase. This can be advantageous to lower the resistance of breathing into the tube. In some embodiments, the breath inlet tube diameter is increased to decrease the space between the inlet tube and collection tube so that breath can interact with the cool wall to collect the condensate. In some cases, the volume of the collection chamber is between 0.5 mL and 50 mL, or a volume between 1 mL and 30 mL, and a volume between 5 mL and 20 mL, or a volume as set out in Table 1 below.

TABLE 1
Examples of collection chamber dimensions

| | | | Cross- | | | |
|---|---|---|---|---|---|---|
| | Internal | Circum- | sectional | | | Surface |
| | Diameter | ference | Area | Length | Volume | Area |
| Name | (cm) | (cm) | (cm²) | (cm) | (mL) | (cm²) |
| 3 mL | 0.83 | 2.61 | 0.54 | 7.50 | 4.06 | 19.56 |
| syringe | | | | | | |
| 3 mL inlet | 0.40 | 1.26 | 0.13 | 7.50 | 0.94 | 9.42 |
| tube | | | | | | |
| 5 mL | 1.18 | 3.71 | 1.09 | 6.00 | 6.56 | 22.24 |
| syringe | | | | | | |
| 5 mL inlet | 0.64 | 2.01 | 0.32 | 6.00 | 1.93 | 12.06 |
| tube | | | | | | |
| 10 mL | 1.45 | 4.56 | 1.65 | 6.70 | 11.06 | 30.52 |
| syringe | | | | | | |
| 10 mL inlet | 1.00 | 3.14 | 0.79 | 6.70 | 5.26 | 21.05 |
| tube | | | | | | |
| 35 mL | 2.29 | 7.19 | 4.12 | 10.47 | 43.12 | 75.32 |
| syringe | | | | | | |
| 35 mL inlet | 1.5 | 4.71 | 1.77 | 10.47 | 18.50 | 49.34 |
| tube | | | | | | |

Typical syringe sizes may be 3 mL or 5 mL. A 5 ml syringe collector has higher collection surface area and lower resistance to breath over 3 ml syringe. The time to collect 50 μL of breath condensate with a −15°, −20°, −30° C. or −40° C. cooling temperature may typically be approximately 15 seconds to collect 50 μL of breath condensate, 30 seconds to collect 100 μL of breath condensate and 60 seconds to collect 150 μL of breath condensate. While difficult to quantify, the resistance to breathing was slight for a collector based on a 3 mL syringe collector but was not noticeable for a collector based on a 5 mL closed end syringe collector. Larger tube collectors and diameters and lengths may collect larger volumes faster. A 10 ml syringe tube collector has higher surface area and lower backpressure, and higher breath liquid volumes may be collected.

Turbulence inducers placed inside the closed end collection tubes of the invention increase the contact of breath to the inside cool wall of the collection tube. In one set of experiments with a collector based on a closed end 5 ml syringe barrel cooled to −15° C., a helical turbulence inducer insert was tested compared to a straight straw insert. The turbulence inducer consists of a hollow tube with an internal diameter of 6 mm and external diameter of 8 mm, and a length of 65 mm. The mouth inlet was included in the design and produced by 3D printing. The external surface was a helical baffle extending 2 mm out, making 11 coils from the base of the tube to the tip. The base was attached directly to a mouthpiece, a tube with internal diameter of 12 mm and external diameter of 14 mm, and 40 mm long. The point of attachment between the mouthpiece and turbulence inducer includes wedge-shaped buttresses which allow the turbulence inducer to firmly press into place within the syringe. The tip includes four triangular vents to allow breath to freely disperse through the end of the syringe.

The helical design performance was compared to 6 mm inside diameter straight walled inlet straws. The 5 ml syringe collector was tested with 3 different turbulence conditions with 15 seconds of breath at approximately −15° C. Two trials with the helical turbulence inducer fully inserted yielded an average breath condensate of approximately 60 μL. Two trials of a straight wall straw fully inserted into the syringe barrel yielded on average 30 μL. Two trials of a straight wall straw inserted just past the opening of the syringe barrel yielded an average of 15 μL breath condensate. Thus, yields were increased with the introduction of turbulence of air passing by the cool surface of the collection barrel tube.

There are several different turbulence inducer designs, all of which were found to outperform a straight walled straw. These include helical with in channel disruptors, open chevon, staggered protrusions and random protrusions. In one design, the open chevron turbulence inducer had the same general dimensions as the helical design, but rather than a helix extending from the outer surface there were a series of broken chevron shapes. Each of these consists of a pair of wedges about 3 mm long. These wedges are arranged to direct breath in a turbulent path against the cold surface of the syringe, while permitting liquid to flow easily toward the tip of the syringe. There were 4 rows of these broken chevrons arranged from the base to the tip of the turbulence inducer, with 8 broken chevrons in each row.

Each version of the turbulence inducer was designed to achieve and balance two primary goals. First, the overall dimensions should minimize the back-pressure produced when blowing through the device. Second, the various baffles and flanges should disrupt the flow of air enough to create turbulence and maximize contact of the breath with the cold outer surface. In these embodiments, to minimize back-pressure, the cross-sectional area of the inner tube was approximately half of the entire cross-sectional area of the syringe. This allowed air to travel through a channel with consistent overall width.

The helical design directs the breath in the longest possible path along the surface of the syringe, thus maximizing opportunities for condensation. Because the turbulence inducer does not form an air-tight fit, a portion of the breath was able to pass over the helical baffles. This further encouraged the air to come into contact with the cooling surface, and for the breath to condense. In some versions of the helical design, wedge-shaped protrusions redirected a portion of the airflow from its smooth helical path. This caused the airstreams to interact with each other and form turbulence, again increasing contact between the air and cold surface.

In another design, vertical and horizontal baffles protrude from the exterior of the tube. In each case, there was an open channel for air to pass through, and this path was long and circuitous. The baffles were sloped in such a way as to encourage a significant portion of the air to pass around them, coming into direct contact with the cold wall.

The design of the turbulent inducer may enhance centrifugal force collection of liquid. The broken chevron design was one design suitable for coalescence and collection of captured breath liquid with a centrifuge, as it allows both turbulent airflow from the tip of the syringe to the opening and unimpeded waterflow from the opening to the tip.

In one example, the collection chamber is devised from a 3 ml syringe barrel with an 8.3 mm internal diameter, cross sectional area of 0.54 cm², surface area of 19.56 cm² and volume of approximately 4.06 mL. The inlet tube has an internal diameter of 0.4 cm, external diameter of 0.54 cm. The turbulence inducers consist of flanges extending outside the inlet tube to a width of 0.72 cm.

In another example, the collection chamber is a 5 ml syringe barrel with an internal diameter of 1.18 cm and internal volume of 6.8 mL with a cross sectional area of 1.09 cm². The collection chamber's internal surface area is 22.24 cm². The inlet tube and turbulence inducer surrounding the inlet tube inserted within the collection chamber occupies approximately half of this volume. The internal volume of the central airway is 1.9 mL with a cross-sectional area of 0.32 cm². The inlet tube has an outer diameter of 7.9 mm and turbulence inducer flanges protrude to 10.75 mm. The cross-sectional area of the collection tube outside of the turbulence inducer is therefore slightly larger than the cross-sectional area inside the turbulence inducer's central air passage. This difference in cross-sectional area compensates for the increased turbulence in airflow once the breath leaves the central air passage and allows breath to flow easily and contact the cold surface without back pressure. Due to the ease of use and speed of capture, this is used in many of the examples of the invention.

In another example, a 10 ml syringe barrel serves as the collection chamber with an internal diameter of 1.45 cm, cross-sectional area of 1.65 cm², surface area of 30.52 cm² and 11.06 mL of actual volume. The inlet tube for this instance has an internal diameter of 1.0 cm and cross-sectional area of 0.79 cm². Flanges on the turbulence inducer extend to 1.4 cm to induce turbulent breath flow.

In another example, a 35 mL syringe barrel serves as a collection chamber. This syringe barrel has an internal diameter of 2.29 cm, cross-sectional area of 4.12 cm², volume of 43.12 mL, and surface area of 75.32 cm². In this example, the inlet tube has an internal diameter of 1.5 cm, cross sectional area of 1.77 cm², volume of 18.5 mL and surface area of 49.34 cm². This larger format option has lower initial efficiency than smaller versions, but experiences less decline in efficiency over multiple minutes of collecting breath.

In the device of the present invention, breath is conveyed through a tube-like fixture or straw to a freezing capture surface. The tube or straw inlet is effectively shielded from ambient air until breath can be introduced and presented into the device. This can be accomplished by having a barrier on the end of the straw or by simply having the straw long enough so that ambient air does not easily enter the device.

In one embodiment of the device, the capture surface is a tubular vial with a means of collecting the liquid from the sample when removed from the freezing source.

The freezing vial, tube or surface of the present invention apparatus is flat or curved, etc., smooth or rough and may contain grooves, baffles or depressions to facilitate collecting liquid. In some cases, the tube may be metal, glass or plastic and the wall thickness of the vial or tube may be 5, 3, 2, 1, 0.05 mm or less. The outside of the vial is cooled, and breath is introduced inside of the tube or vial. A disposable straw or tube inlet may be used to introduce the exhaled breath to the vial, whereupon a sample is collected in the vial. The freezing surface of the vial is protected from ambient air until breath is introduced. The shield is removed, and breath frozen and liquid condensate is collected.

After capture, the collection of liquid into the vial at the closed end can be performed with a scraping plunger or with a centrifuge. Where a plunger is used with a collecting device in which a vial seals the end of a syringe barrel, the plunger must be configured to allow air to escape as the plunger is depressed or inserted into the barrel. Using a centrifuge may cancel the need for a special plunger design. Insertion and processing the collected breath condensate with a centrifuge will move liquid to the collection vial and displace any air. No scraping with a plunger is necessary with application of centrifugal force. In some embodiments, the turbulence inducer does not need to be removed and the tube may be centrifuged directly to collect the liquid at the closed end vial.

Several custom centrifuge rotors were tested to collect exhaled breath condensate. Three different mounts were designed, and these were rotated on 3 different rotors. In the rotors tested in these experiments, the closed end capture tubes were on the same plane. In other embodiments, the capture tubes may be at an angle for easier insertion into the centrifuge. One battery powered rotor rotates at approximately 500 rpm, one hand cranked rotor can rotate at approximately 1000 rpm, and one AC powered rotor rotates at approximately 4,000 rpm. The mounts to hold syringes in place on the centrifuge rotor can have at least three basic arrangements. In one arrangement, the two syringes were each held in place 75 mm from the center of the mount, directly across from one another, and aligned with each other. In another arrangement, the syringes were mounted off-center, allowing them to be loaded more easily without interfering with each other. In this arrangement, the base of the syringe was 5 mm from the center of the rotor in the direction that the syringe was pointing and shifted 15 mm laterally. In the third arrangement, the syringes were mounted in a vertical stack facing opposite directions. The base of each syringe was mounted 5 mm from the axis of rotation.

Each mount design was tested on the battery powered, 500 rpm rotor. 100 μL of water were distributed along the length of the turbulence inducer before inserting it into the syringe. Ten trials were then spun for 10 seconds with each style of mount; in-line, off-center, and vertically stacked. Then the water collected in the vial was measured. The vertical stack averaged 89.1 μL collected, off-center averaged 88.5 μL collected, and in-line averaged 94.7 μL collected. The difference between the in-line arrangement and the other two is statistically significant, although the difference between the vertical stack and off-center arrangement is not statistically significant.

Due to its geometry, the force at the base of the syringe is 15 times greater in the in-line arrangement than the other two, although there is only a twofold change at the tip. For bench-top convenience, a more compact, easy to load design such as the side-by-side arrangement may be desirable, although the in-line arrangement may be more effective.

In tests with exhaled breath condensate rather than manually adding droplets of water, the battery powered motor with the side-by-side mount performed less efficiently, capturing less than half of the exhaled breath. The rotor with a more powerful AC motor was substituted to be able to spin at 4000 rpm, an eightfold increase in speed. This allowed for 64 times the centrifugal force, which is sufficient to collect >99% of the exhaled breath condensate. The comparison is shown in Table 2.

TABLE 2
Comparison of collection amounts for
different centrifuge mount styles

| | Vertical Stack | Off-center | Straight-line |
|---|---|---|---|
| | 89 | 77 | 95 |
| | 80 | 82 | 92 |
| | 91 | 92 | 97 |
| | 88 | 91 | 98 |
| | 90 | 93 | 90 |
| | 81 | 90 | 96 |
| | 87 | 91 | 94 |
| | 99 | 90 | 95 |
| | 94 | 85 | 98 |
| | 92 | 94 | 92 |
| Mean | 89.1 | 88.5 | 94.7 |
| Std Dev | 5.66 | 5.44 | 2.71 |

Materials collected in the frozen and/or liquid condensate of breath may include virus, bacteria, spores, organic compounds, volatile inorganic compounds, proteins, and any other biological compound or material.

The collected breath liquid may be analyzed to detect nucleic acids. This may be performed by amplification or tagging. They may be quantified by various methods including RPA, LAMP, PCR, qPCR, RT-qPCR and any other detection device including next generation sequencing. These detection methods may segment the sample and analyze the segments individually. Other detection devices and methods include lateral antigen, mass spectrometry, LC/MS, UV, IR, etc. Applications of the technology include detection of viral or bacterial infections spread by exhaled pathogens, as by definition the subject would be infectious if these are present in the breath or the detection of organic molecules. The device may be used as a tool for diagnostics or research. The nucleic acid samples may be RNA or DNA. Preferably, the target nucleic acid is viral, for example where the virus is selected from the group consisting of COVID-19 (caused by Severe Acute Respiratory Syndrome Corona Virus-2, SARS-COV-2), Acquired Immune Deficiency Syndrome (AIDS, caused by Human Immuno-deficiency Virus, HIV), cold sores, chickenpox, measles, flu, influenza, some types of cancer and others. Other examples include Herpes simplex, varicella-zoster virus (VZV), Respiratory syncytial virus (RSV), Epstein-Barr virus, Cytomegalovirus (CMV), Coronaviruses, Rotavirus, Hepatitis, Monkeypox, Marburg, Genital warts (human papillomavirus, or HPV), and BK virus. Examples of bacteria that may be detected using the present invention include tuberculosis (TB) or staphylococcus.

In addition to liquid collection from breath, liquid may be collected from ambient spaces. Air may be pumped through the device to collect and detect materials that may be present in the ambient air of a room or building or even outside a building.

It is possible to capture and detect a virus directly without lysing or sample preparation. The freezing breath collection may keep a virus stable, can capture all chemicals, and can be done rapidly. The capture and processing can be done reproducibly because greater than 70%, greater than 80% or greater than 90% of the virus, bacteria or chemical can be collected. All of the sample can be processed and detected. Some portion of the virus can release the nucleic acid which can be detected. However, adding an organic solvent, acetonitrile for example, will kill or inactivate the virus so that the collected liquid is safe to handle. Methods, devices and kits useful for the processing of nucleic acid samples for storage and analysis, especially by amplification techniques, are described in our co-pending publication WO 2021/209564 (PCT/EP2021/059815 filed on 15 Apr. 2021), the whole content of which is incorporated by reference in its entirety.

Column sample preparation for nucleic acid can be used. Enzyme degradation of the virus protein can be used to release the nucleic acid before detection. In other approaches, the detection methods can involve essentially no sample preparation and the nucleic acid may be detected directly from virus or other materials containing nucleic acid. Other organics can be detected directly using mass spectrometry and other methods.

Analysis of the samples may be automated. The sample may be introduced directly into mass spectrometer or LC-MS, micro volume UV spectrometer or other light absorbing spectrometer. Nucleic acid detection requires only lysing of the virus and bacterial detection with an organic liquid such as acetonitrile. Detection may be with LAMP, RT-PCR, LC, LC-MS, GC, GC-MS, MS, IR, UV, FTIR, NMR or any analytical technology. Detection may be performed with Loop-Mediated Isothermal Amplification, Whole Genome Amplification & Multiple Displacement Amplification, Strand Displacement Amplification & Nicking Enzyme Amplification Reaction, Helicase-dependent Amplification, Recombinase Polymerase Amplification and SIBA Nucleic Acid Sequenced Based Amplification and Transcription Mediated Amplification.

In order to be most useful and provide a safe margin of infectiousness, the methods and devices of the present invention may detect a ten-fold lower viral shed rate than would be likely to cause infection for a given situation. For example, a teacher or student in a school would be infectious if they are shedding about 600 viral particles per minute. The methods could be used to collect 30 seconds of breath from each student and assess viral load. If that viral load is more than 300, the subject is considered infectious, therefore the present invention would report anything above 30 viral particles from this sample. For the most sensitive situations, such as plane or train travel, the methods of the present invention could test a full minute of breath and detect as little as 5 viral particles.

While many LAMP studies have shown a Limit of Detection (LOD) in the range of 100 viral particles, various techniques are available to increase this sensitivity to the level of detecting 2-3 viral particles. The present invention can incorporate state of the art techniques including fluorescent detection to increase sensitivity and specificity. Novel viral lysing agents, such as acetonitrile, will improve recovery of viral nucleic acid and further increase sensitivity.

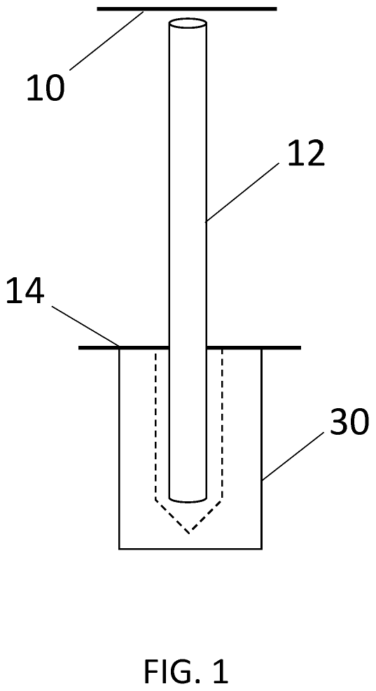

FIG. 1 shows an example of a breath collection device that can be used in embodiments of the present invention. In this example, the collection device comprises a collection vial 30 that is suitable for insertion into a receptacle that holds a coolant undergoing an endothermic process. Collection vial 30 can be metal or plastic or another material or combination of materials. Breath is exhaled and introduced into the collection device through an inlet tube (e.g. straw) 12, whereby liquid aerosol particles and vapor are captured within collection vial 30. Ambient air is shielded from the interior of the vial by barrier 10 and freezer cover 14.

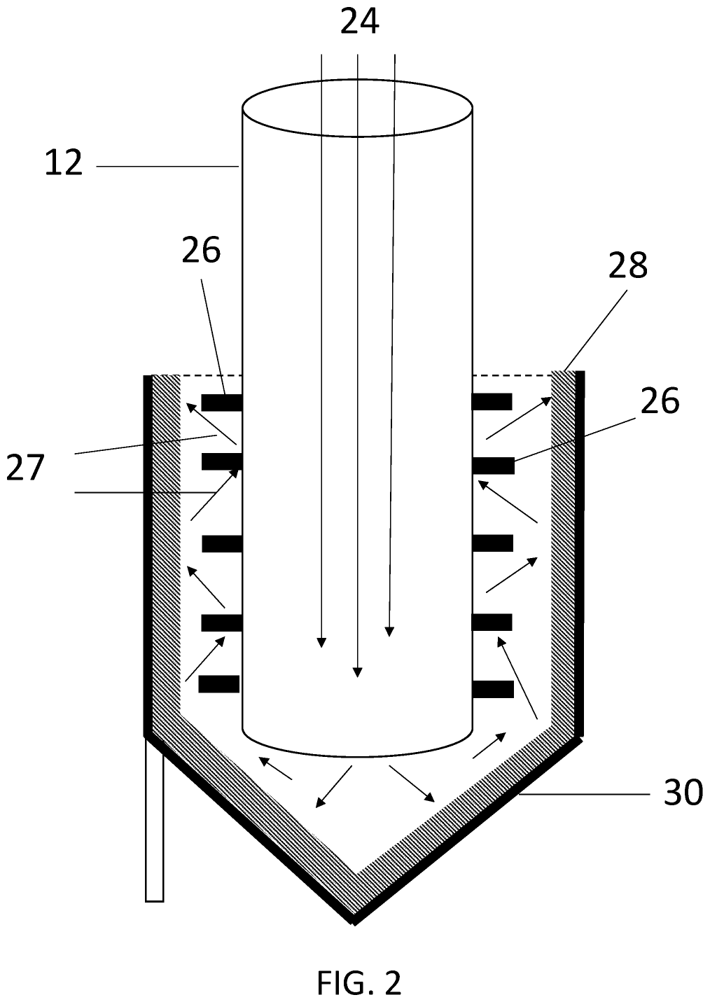

FIG. 2 shows how breath flowing through the inlet tube 12 in direction 24 is introduced into collection vial 30. The air flow reverses at the bottom of the vial and then moves along the wall of collection vial 30. The flow may be laminar. Turbulent air flow increases collection speed and amount. Turbulence inducers 26 may be positioned on the outside of inlet tube 12 as shown here or may be positioned on the inside surface of collection vial 30. The turbulent flow directions are indicated by arrows 27 and may be straight and/or random or may incorporate swirl motions in different directions. Ice and liquid 28 are collected on the inside of collection vial 30 at the wall.

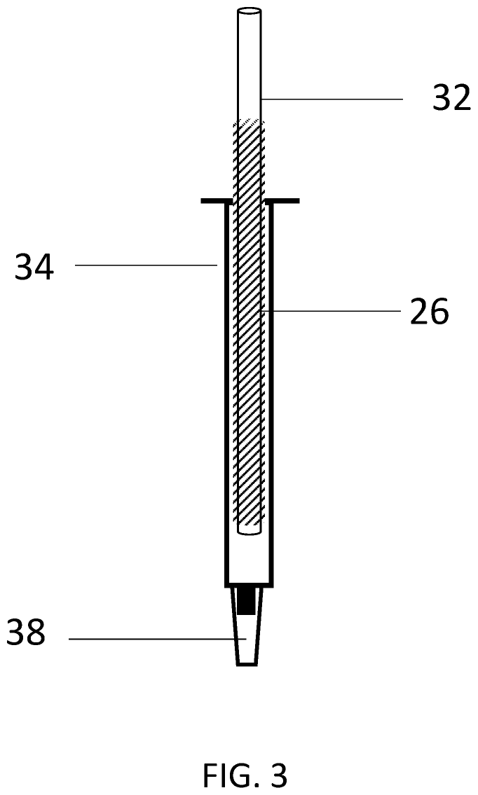

FIG. 3 shows another example of a breath collection device that can be used in embodiments of the present invention. In this example, the collection device is formed from syringe barrel 34 having a vial with a closed end 38 mounted at a distal end thereof. This structure essentially makes the entire syringe barrel a vial arranged to collect liquid at its distal end. The device is suitable for insertion into a receptacle that holds a coolant undergoing an endothermic process as discussed in more detail below. Breath is introduced to the interior of the syringe barrel 34 through a vertical tube 32. The inlet of vertical tube 32 may be round to place the lips around a tube or may be a mouth piece into or onto which the mouth may be placed. Air breath flow into tube 32 reverses near the end of closed end vial 38 and passes through turbulence inducers 26 to increase contact with the inside wall surface and space of syringe barrel 34 and closed end vial 38. The frost and liquid can be collected by gravity drainage, solvent washing, centrifugal force or a plunger may be inserted in syringe barrel 34. For example, a rapid plunger scraping motion may be used to force liquid into closed end vial 38. If a rapid plunger motion is used, closed end vial 38 may be loosened to allow air venting without allowing liquid to escape.

The syringe barrel 34 may contain a turbulence inducer. In one example, the external surface of the turbulence inducer may be a screw-like helix, directing the breath along the longest possible route back out of the syringe barrel. The helix does not fit snugly against the inside of the syringe barrel, allowing some breath to leak past, which brings this air into direct contact with the freezing surface. This iteration may be twisted upon removal to minimize accidentally removing breath condensate. In another iteration, a series of baffles with small vents direct the breath toward the freezing surface, or to make sharp right angle turns, increasing turbulence. In another iteration, baffles are slanted at an angle resembling a herringbone pattern. This pattern again directs exhaled breath toward the freezing surface, while also generating pockets of high and low pressure, thus inducing turbulence. In this iteration, the turbulence inducer may remain in the syringe while it is centrifuged, and condensed liquid will be directed toward the tip.

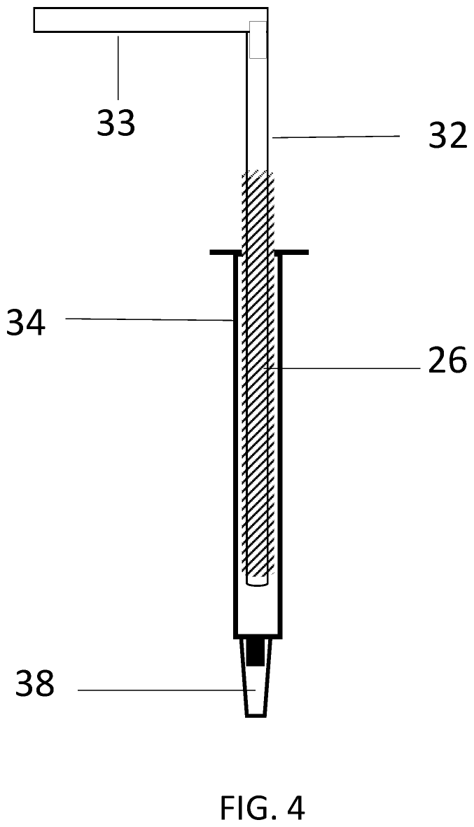

FIG. 4 another example of a breath collection device that can be used in embodiments of the present invention. This device has all the same features as those shown in FIG. 3 except that a horizontal breath inlet tube 33 is used instead of a vertical inlet. The horizontal inlet tube may include a liquid trap (not shown). The entrance of horizontal breath inlet 33 may be round to place the lips around a tube or may be a mouth piece into or onto which the mouth may be placed.

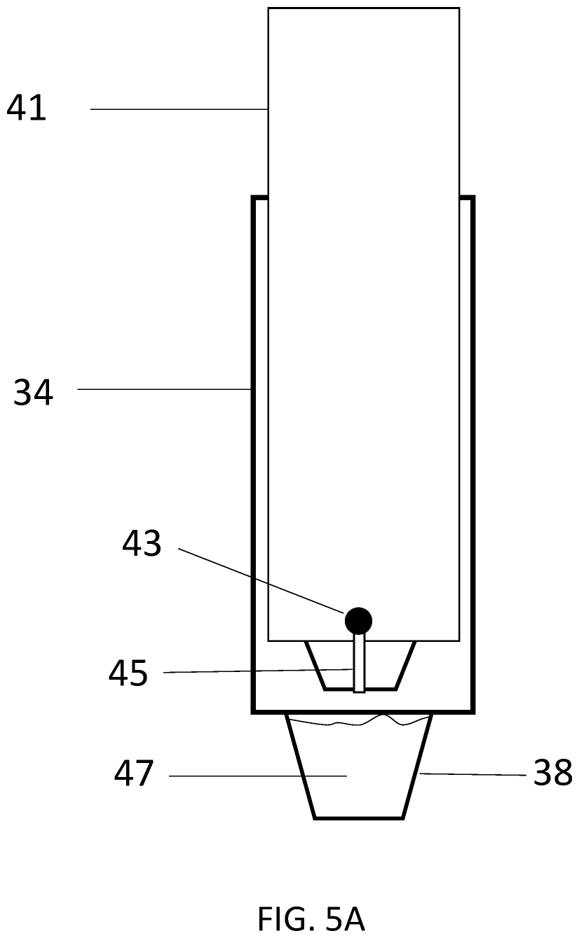

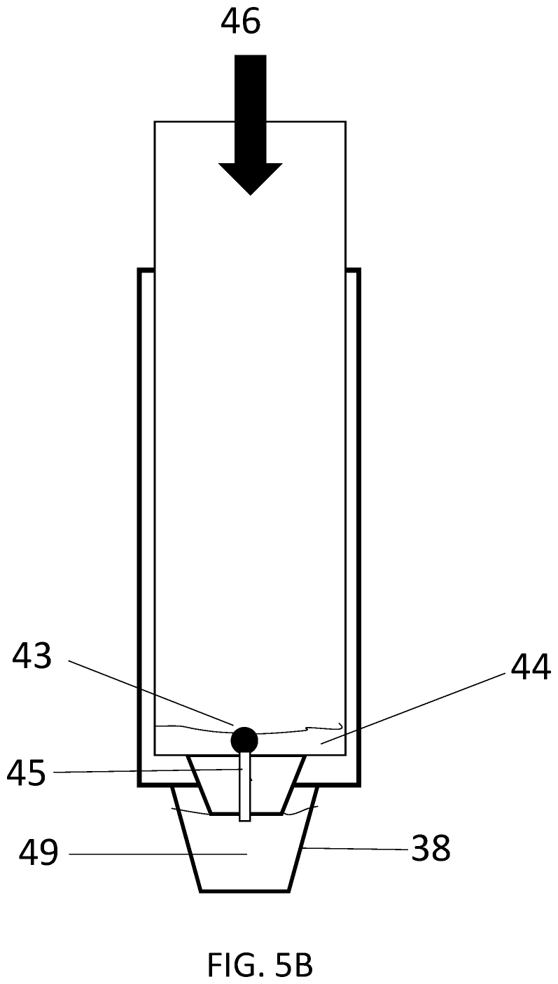

FIGS. 5A and 5B show a device used to normalize the volume of liquid collected in a syringe barrel. FIG. 5A shows modified plunger 41 collecting liquid 47 from syringe barrel 34 into closed end vial 38. Relief tube 45 with top check valve 43 allows air to pass into modified plunger 41 as it is inserted. Collected liquid 47 may be scraped to drain into closed end vial 38. FIG. 5B shows further downward motion 46 of modified plunger 41 into syringe barrel 34 and closed end vial 38. The relief tube 45 with top check valve 43 now allows liquid to pass through the plunger 41 as it is inserted without permitting liquid to pass in the opposite direction. Excess liquid 44 passes through the plunger, leaving only pre-determined volume of collected liquid 49 in closed end vial 38. Closed end vial 38 may be removed to access liquid for further processing.

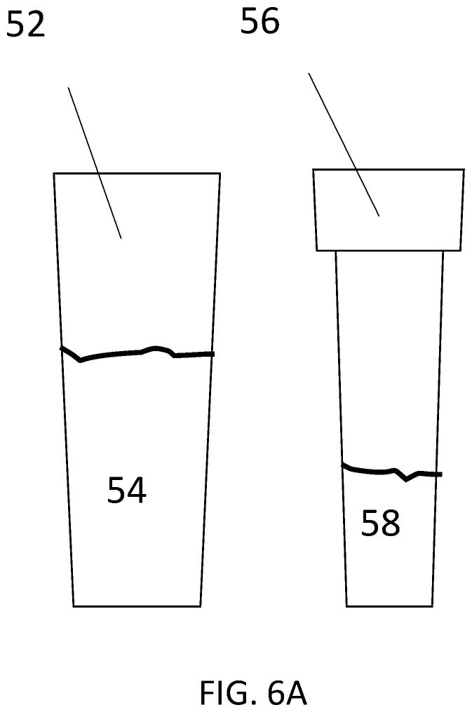

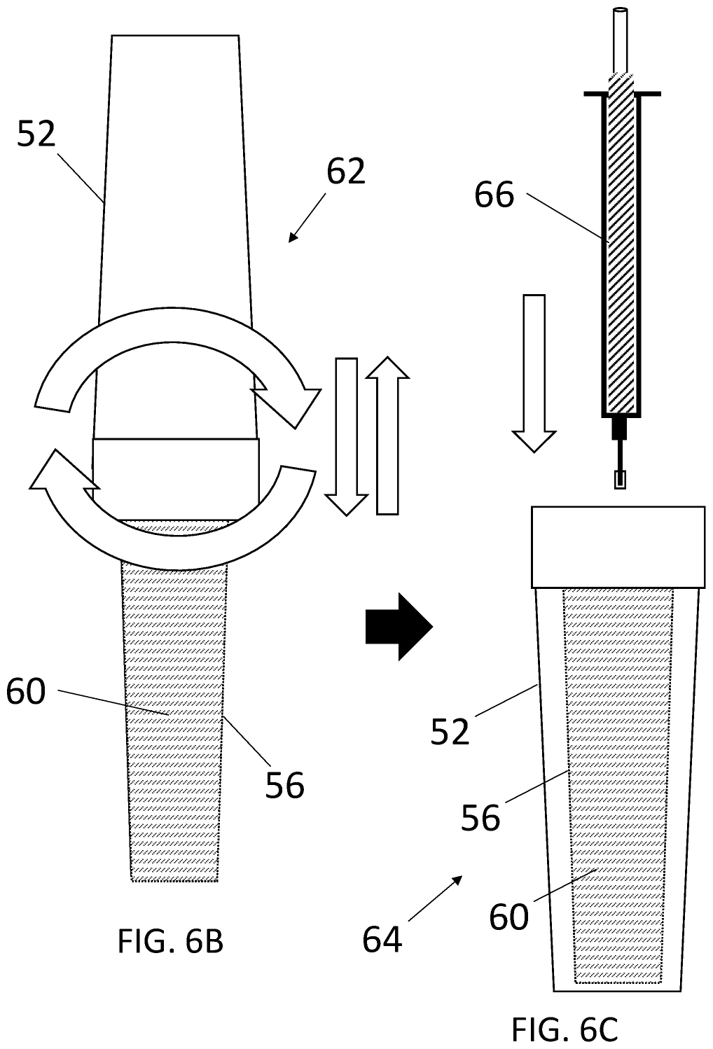

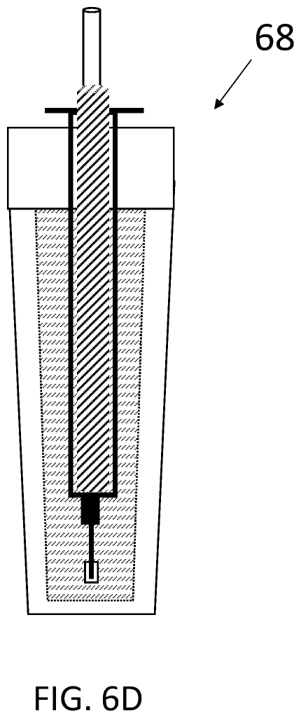

FIGS. 6A to 6D illustrate the components, assembly and use of a collection device that is an embodiment of the invention. In the specific example presented here, a salt ice coolant mixture is used. However, it can be understood that any of the endothermic coolants or coolant mixtures discussed herein may be used with this collection device.

FIG. 6A shows components for preparing a coolant mixture. In this example, the components comprise two tubular sections 52, 56 that each contain one or more components of the coolant mixture. The tubular sections 52, 56 are connectable in an opposed configuration to form a connected assembly that can be used as a mixing vessel that defines an enclosed mixing volume in which the components are combined. The connected assembly can be shaken to mix the coolant elements. In this specific example, ice 54 is provided as a first component of the coolant mixture. The ice 54 is contained in a first mixing tube 52. Salt 58 in powder form is provided as a second component of the coolant mixture, and is contained in second mixing tube 56. In this example, the second mixing tube 56 is smaller than the first mixing tube 52 so that it can fit within the first mixing tube in use (see FIG. 6C). In this example, the top of second mixing tube 56 has a connection interface configured to mate and seal with the first mixing tube 56 to form the connected assembly. The connection interface may comprise any suitable sealing structure. In some implementations, the first and second mixing tube may each comprise engagement features that cooperate to form the connection interface. For example, the engagement feature may comprise a threaded connection or the like.

FIG. 6B illustrates the connected assembly 62 formed by connecting the first mixing tube 52 and the second mixing tube 56. The connected assembly 62 can be agitated (e.g. shaken and inverted) to combine the components to produce a coolant mixture, which in this example is a salt ice mixture 60. The salt lowers the melting temperature of the ice, which causes the natural (endothermic) melting process to draw more heat from the surrounding environment. This process manifests itself as a drop in temperature of the salt ice mixture 60. In the case of using sodium chloride the mixture cools to −18° C. to −21° C. depending on the mixing.

FIG. 6C shows a receptacle 64 that is formed by inserting the second mixing tube 56 containing the salt ice mixture 60 into the empty first mixing tube 52. The first mixing tube 52 provides insulation that assists in maintaining the temperature of the salt ice mixture. The second mixing tube 56 is dimensioned to receive a breath collection device 66 into the coolant mixture 60. The breath collection device may for example be any of the devices discussed above with reference to FIGS. 1 to 4. In FIG. 6C itself, the breath collector device 66 comprises a disposable plastic 5 ml syringe barrel having a vial with a closed end mounted at a distal end thereof.

The receptacle 64 may comprise a lid for retaining the breath collector device 66. The lid may for example be a cover that snaps on to mouth of the receptacle 64.

FIG. 6D shows the assembled device 68 ready for breath collection. The coolant mixture is in direct contact with the outer wall of the syringe that forms part of the breath collector device 66. The syringe may have a closed end where condensate can be collected and subsequently directed into a vial or through a needle.

The entire collection device is preferably a single use article intended to be disposable as refuse. As such it is desirable that it is substantially devoid of metal material. The collection device, i.e. the first and second mixing tube and (if different) the receptacle may consist of paper or plastic materials.

For example the first mixing tube 52 may be a tubular plastic container having a height of 10 cm and an average diameter of 3 cm. In use it may contain 45 mL or 30 g of crushed ice. In this example, the second mixing tube 56 may have a shape that complements the first mixing tube. For example the second mixing tube 56 may be a tubular plastic container having a height of 10 cm and an average diameter of 1.5 cm. In use it may contain 15 mL or 10 g of sodium chloride powder.

In another example, the receptacle 64 may comprise a pair of nested paper cups, e.g. of differing sizes such as 8 ounce (240 ml) and 12 ounce (360 ml) filled with ice.

In a further example, the coolant mixture (e.g. ice and salt) may be combined in a disposable 50 ml conical tube attached to a secondary polypropylene cylindrical container. The container may have a height of 100 mm and a diameter of 38 mm with an internal volume of approximately 90 mL. The secondary container may be slightly shorter and wider than the 50 mL tube. The secondary container may include a threaded top which screws into the top of the 50 ml conical tube to create a single sealed container of approximately 140 mL volume. The secondary container may include a line indicating 12 mL of volume to measure an appropriate amount of sodium chloride.

In use, the 50 ml conical tube is filled with crushed ice, and the 90 mL container has sodium chloride added to the 12 mL fill line. The two tubes are screwed together to form a single sealed container and shaken vigorously for approximately 30 seconds. The resulting saltwater slush is gathered in the 50 mL tube and the secondary tube is unscrewed and slid over the bottom of the 50 mL conical tube. This allows the wider tube to serve as an insulator, allowing the tube to be held comfortably while the internal slush reaches −18° C. or lower. This sleeved assembly forms a receptacle for receiving a breath collection syringe.

The receptable may be closed using a screw top lid that is provided with a suitable aperture for receiving and supporting the breath collection syringe.

Because ice is less dense than water, and crushed ice may contain air pockets, the 50 mL of crushed ice reduces to approximately 35 mL as it melts. When the breath collection syringe is inserted into the slushy saltwater, it displaces the water and raises its level to cover the vast majority of the syringe. This allows efficient heat transfer from the syringe to the saltwater slush, and when breath is introduced a large proportion of the water vapor is captured as breath condensate.

The coolant mixture can be dry ice, water ice with sodium chloride, calcium chloride hexahydrate, or any other salt producing a cooling endothermic process. The temperatures reached are dry ice (−60° C.), ice-sodium chloride (−18° C.) and ice-calcium chloride hexahydrate (−31° C.). With the collection device configurations discussed above, these temperatures can allow for suitable volumes of condensate to be obtained in 15-30 seconds depending on mixing. Coolant mixtures with dry ice have been found to provide rapid cooling and result in large condensate volumes.

Several salts may be added to ice in different ratios to create a cold bath. Shown in Table 3 below are several examples of coolant mixtures suitable for use in the present invention.

TABLE 3
Examples of cooling mixture composition
and associated temperatures

| Mixture temperature | |
|---|---|
| (° C.) | Coolant mixture composition |
| 0 | water + ice |
| −2 | water + ammonium chloride |
| −12 | water + sodium nitrate (III) |
| −15 | water + ammonium thiocyanate |
| −15 | ice + sodium acetate |
| −18 | ice + ammonium chloride |
| −21 | ice + sodium chloride |
| −25 | ice + ammonium nitrate (V) |
| −28 | ice + sodium bromide |
| −30 | ice + potassium chloride |
| −33 | ice + magnesium chloride |
| −37 | ice + sulfuric acid (VI) 66% |
| −40 to −55 (depending | ice + calcium chloride hexahydrate |
| on mass ratio) | |

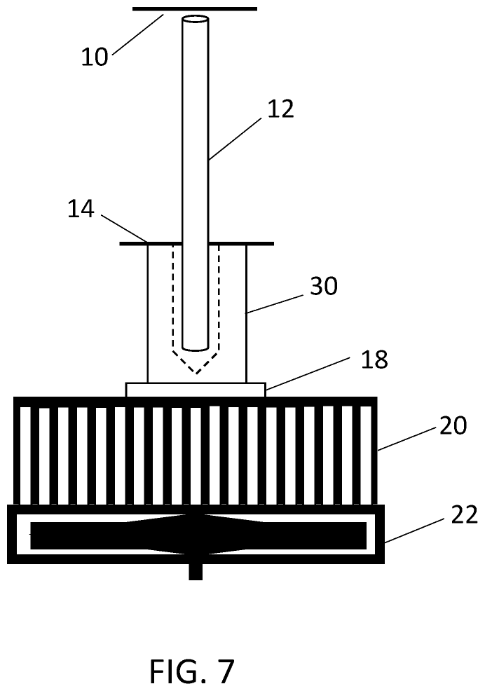

FIG. 7 is a schematic diagram showing a breath collection apparatus that uses an active cooling system (which in this example is a Peltier device) for cooling and freezing a vial in the collection device of FIG. 1. Ambient air is shielded from the device by barrier 10 and freezer cover 14. Collection vial 30 is cooled directly by a Peltier device 18. Collection vial 30 can be metal or plastic or another material or combination of materials. Breath is exhaled and introduced into the device by straw 12 and liquid aerosol particles and vapor are captured in collection vial 30. The device super cools the walls of collection vial 30 so that the sample is collected quickly. However, the capacity of the device is limited. Heat sink 20 draws heat away from the hot side of the Peltier device, and fan 22 blows air to cool the heat sink to ambient temperature.

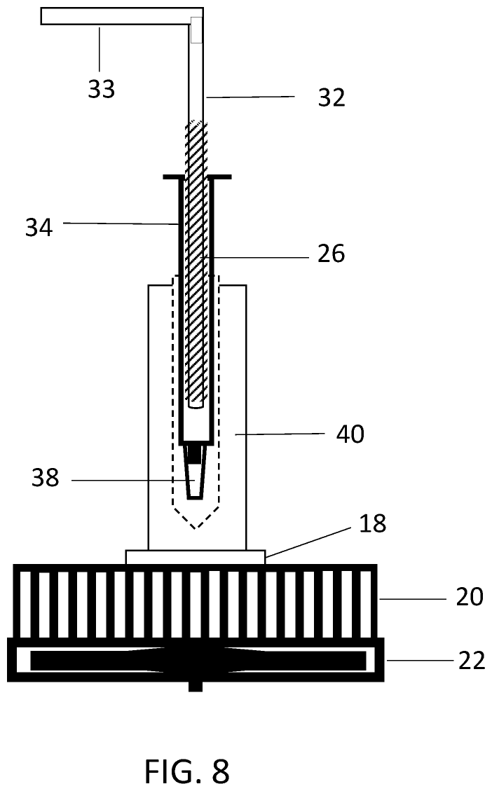

FIG. 8 is a schematic diagram showing a breath collection apparatus that uses an active cooling system (which in this example is a Peltier device) for cooling and freezing a syringe barrel of the collection device shown FIG. 4. FIG. 8 shows a device formed from syringe barrel 34 and vial with a closed end 38 essentially making the entire syringe barrel a vial with collection of liquid at the end. Copper or metal tube 40 is placed on top of Peltier device 18 to cool syringe barrel 34 and closed end vial 38. Breath is introduced with vertical tube 32. The inlet of vertical tube 32 may be round to place the lips around a tube or may be a mouth piece into or onto which the mouth may be placed. Air breath flow into tube 32 reverses near the end of closed end vial 38 and passes through turbulence inducers 26 to increase contact with the inside wall surface and space of syringe barrel 34 and closed end vial 38. The frost and liquid can be collected by gravity drainage, solvent washing, centrifugal force or a plunger may be inserted in syringe barrel 34. For example, a rapid plunger scraping motion may be used to force liquid into closed end vial 38. If a rapid plunger motion is used, closed end vial 38 may be loosened to allow air venting without allowing liquid to escape. Heat sink 20 and fan 22 are as shown in FIG. 7.

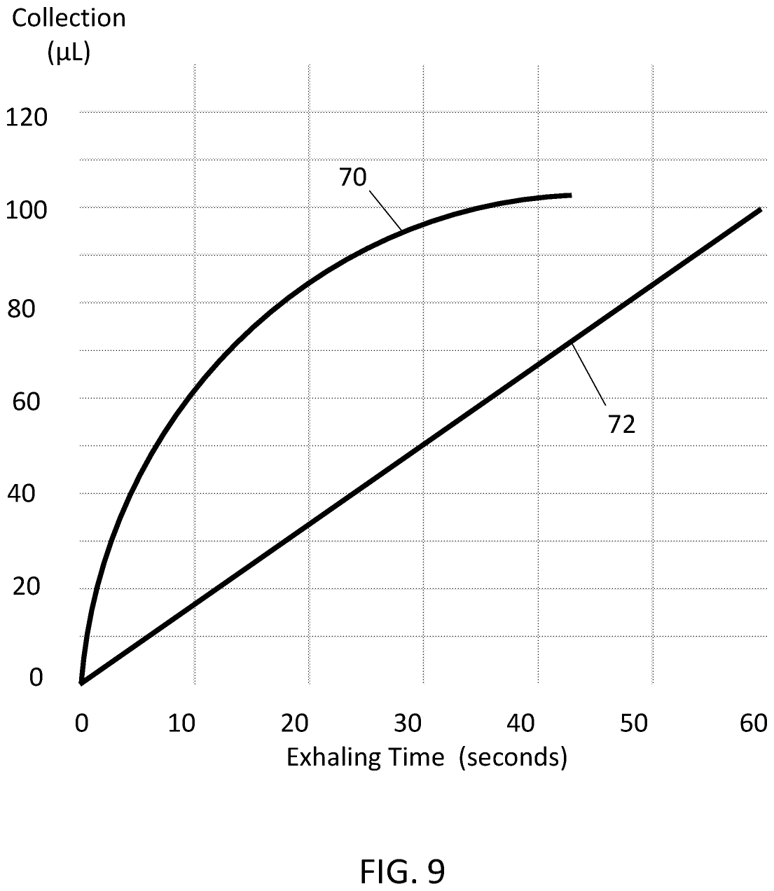

FIG. 9 compares the collection of liquid from the syringe and vial of the invention in curve 70 compared to conventional EBC collection tube which is shown in line 72. The figure illustrates the difference of collection of liquid aerosol particulate and liquid vapor on the device of the invention vs. the conventional device. Conventional devices have a large capacity to capture breath as liquid but also need this capacity to be able to collect and process the liquid. The device and method of the invention collects rapidly but because the device is small, it also quickly lowers the rate of collection. The figure shows the exponential collection of the present invention 70 vs. linear collection of conventional devices 72. The total amount of liquid that can be collected with the device of the invention is low, but sufficient liquid can be collected quickly. Exponential collection is rapid at first and then levels off. Rapid collection is possible, but collection quickly slows due to the insulating ice and liquid from the collected breath or low surface area. In addition, the surface needs to be shielded to prevent ambient liquid vapor from being collected as ice and preventing or slowing further capture of breath liquid and vapor. Because the capacity of the device of the invention is small relative to devices of the prior art, R-tube and other devices, capture of liquid from ambient air will lower the ability to capture additional breath liquid particles and vapor.

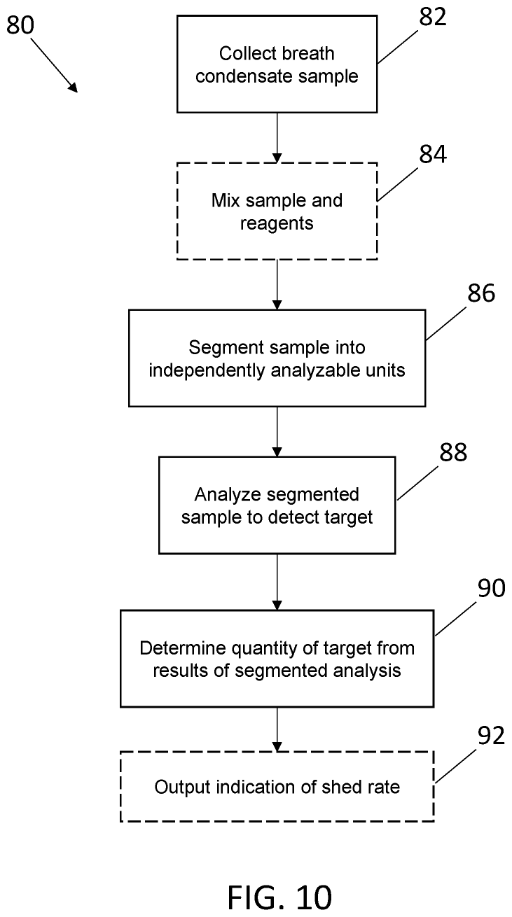

FIG. 10 is a flow diagram showing steps in a method 80 for determining a quantity of a target in a breath condensate sample that is an embodiment of the invention.

The method 80 includes a step 82 of collecting a breath condensate sample, for example using any of the collection devices discussed above. The sample may be collected over a predetermined amount of time (referred to herein as a sampling duration), which may be a minute.

The method 80 may proceed with an optional step 84 of mixing the sample with detection reagents. For example, one or more detection reagents may be added to the sample, e.g. in the collection chamber in which it is held. In one embodiment, an organic solvent may be added to lyse a virus and release the virus RNA. In one embodiment, nucleic detection reagents including amplification reagents may be added to the sample mixture.

The method 80 continues with a step 86 of segmenting the sample into a plurality of independently analyzable units. For example, the sample (or the mixture of sample and detection reagents) may be physically separated into independent digits or segments. The independent segments may comprise droplets, wells, separate areas of filter media, etc. The independent segments may each be retained in a suitable liquid-retaining structure, e.g. well, or the like. The segmenting step 86 may comprise transporting the sample from the collection device (e.g. vial or syringe barrel) to an array of liquid-retaining structures. For example, the sample may be distributed across the array of liquid-retaining structures using a centrifuge, plunger or other fluid delivery mechanism.

After the sample is segmented, the method 80 continues with a step 88 of analyzing the independent units to detect for the presence of the target. In embodiments where the target comprises a nucleic acid, the analyzing step may include amplifying the nucleic acid. Amplification may be performed by thermal cycling or isothermal. There are generally two types of nucleic acid amplification detection: endpoint and real time. In addition, pathogen detection may be directed toward individual pathogen entities. Real time amplification and detection is when the amount of nucleic acid or pathogen is measured as the detection method proceeds. Real time detection can be performed with any detection method including qPCR, qLAMP, qRPA, etc. Isothermal detection may be performed by LAMP, RPA or any other detection method. Pathogens may be detected by any method where each independent unit (individual segment) can be analyzed to detect presence of the target.

As discussed in more detail below, the results of the analysis are used in a subsequent step 90 to determine a quantity of the target in the original sample, e.g. by calculating an amount of pathogens that have been shed during the sample collection process. The nature of the segmentation and the information that can be reported depends in general on a balance between three parameters:
1) a range of concentration or number of viruses or pathogens in the sample that it is desirable to detect, which would normally be a range associated with a shed rate indicative of infectiousness,
2) a number of independent analyzable units, and
3) a probability that each independent analyzable unit has only one pathogen.

The analysis step may output, for each analyzable unit, a binary indication of whether or not the target is present in that unit. Assuming that the analysis is capable of detecting the presence of a single pathogen in an analyzable unit, it follows that an absolute detection limit corresponds to a scenario in which a (single) pathogen is detected in only one of the plurality of independently analyzable units. If a sample were collected in 20 seconds, the detection limit for shed rate in the above scenario would be 3 pathogens/min. The three parameters above may be balanced to ensure that a shed rate that is ten times this detection limit (i.e. 30 pathogens/min) is readily determinable.

Similarly, it may be understood that for any given array of independently analyzable units, a maximum detection limit occurs just before pathogen is detected in all of the independent analyzable units, i.e. in the scenario where pathogen is not detected in only one analyzable unit. The parameters above may be balanced such that this scenario occurs at a shed rate that corresponds to an infectiousness state (preferably a highly infectious state) so that the reportable range of shed rate encompasses different states of infectiousness, e.g. low infection risk. medium infection risk, high infection risk, etc.

The method 80 may further comprise a step of reporting the outcome of the analysis. In some examples, this reporting step may give a yes or no answer to indicate whether or not the pathogen is detected. The reporting step may also provide a detection limit for the method, i.e. a threshold number of pathogens above which detection is possible.

In some embodiments, more than just a yes/no infectious answer may be provided. For example, if an individual is not shedding a pathogen, a “no” answer indicates that an individual is not shedding at a rate equal to or greater than the detection limit of the analysis, which may be defined in terms of a minimum detectable shed rate (e.g. number of virus/min). Using this information and information on the ability of the virus to infect for the defined disease, the individual can be said to be non-infectious. However, if an individual is shedding and is infectious, then the amount and rate the pathogen may be used to determine a degree of infectiousness of the individual. The reporting step may include an qualitative indication of degree of infectiousness.

Alternatively or additionally, the method 80 may include an optional step 92 of outputting an indication of shed rate that is calculated using the determined quantity of the target and the sampling duration.

### Using Segmentation to Determine Shed Rate

The total number of copies of a targets from a reaction occurred across multiple sub-volumes can be expressed as follows [1]:

$$N λ = N ln ( N / N - x )$$

where Nλ is the total number of copies across all sub-volumes, N is number of sub-volumes, x is the number of sub-volumes in which a copy is positively detected, and λ is the average number of copies in a sub-volume, where concentration (i.e. copies/μL) can be expressed as number of copies/total volume×dilution factor. We can apply this equation to the segmenting process of the present invention to derive an equation for the maximum copy count that can be quantified for a given number of independently analyzable units (i.e. segments or digits).

Maximum quantification occurs when a single segment has no copies of the target. If there are more copies of the target than this quantity, then all segments will be expected to contain at least one copy of the target, which would be indistinguishable from any higher copy count. The measurement will still be qualitative, however.

Mathematically, if all segments except for one is positive, then N−x=1, and therefore:

$$N λ = N ln ( N / 1 ) = N ln N$$

This equation makes it relatively simple to determine a maximum viral count to quantify for a given number of segments, namely the number of segments times the natural log of number of segments. In other words, a formula for the maximum quantifiable viral count depending on number of independently analyzable units (i.e. segments) can be expressed as

Cₘₐₓ=N ln N

where N is the number of segments, and Cₘₐₓ is the maximum count that can be quantified.

This would be the calculation for the best estimate of viral count in the scenario where all segments except for one were positive for the presence of the target (or a copy thereof). At this point, precision is already declining, but there is no resolution whatsoever at higher viral counts.

The shed rate (which for viruses can also be expressed using the term “viral load”) that is relevant to quantify may vary depending on the pathogen concerned, i.e. on the individual virus or infectious agent. It is desirable to quantify up to a level high enough to be likely to infect under any conditions. For example, Sars-COV-2 is able to infect with a much lower viral count than influenza, therefore anything above 1,000 copies per minute of Sars-COV-2 is highly contagious, whereas influenza may have a higher cutoff. In this case, 100 segments would be sufficient to quantify 460 viral particles from a 20 second sample, therefore able to quantify 1,000 viral particles per minute of shed rate.

TABLE 4
Maximum detectable shed rate for different numbers of segments

| | Max detectable | Max Shed rate |
|---|---|---|
| Segments | count | (pathogen/minute) |
| 16 | 44.4 | 133.1 |
| 49 | 190.7 | 572.1 |
| 96 | 438.2 | 1,314.5 |
| 100 | 460.5 | 1,381.6 |
| 225 | 1,218.6 | 3,655.9 |
| 384 | 2,285.0 | 6,855.1 |
| 20,000 | 198,069.8 | 594,209.3 |

Table 4 shows the maximum detectable count and maximum quantifiable shed rate for various numbers of segments according to the above equation. Note that the last row of the table shows data for 20,000 segments. All commercial digitizing PCR instruments are based on digitizing sample to this value. However, this type of instrument and digitizing is inappropriate and unusable for analyzing breath condensate for maximum detectable count and shed rate.

Depending on the sampling time, the appropriate number of segments used in embodiments of the invention may be equal to or less than 10,000, 5,000, 4,000, 3,000, 2,000, 1,000, 900, 800, 700, 600, 500, 400, 300, 200, 150, 100, 90, 80, 70, 60, 50, 40, 30, 20, 10, 5, 4, 3 or 2.

For COVID, a shed rate around 200-500 virus/minute would be concerning in most situations (classroom, work day, etc.), 13,000 would risk infecting folks even in cases of brief, social distanced contact. So, if a 10× safety margin is considered, 100 segments could be enough for detecting COVID, with anything above 1,300 pathogens/minute being considered so infectiousness that further quantification is not needed.

For influenza, 1,000-3,000 virus/minute is a relevant range for work day, classroom settings, and 90,000 would be highly infectious. To distinguish around that level, there should be 10,000 segments or more if particle counting quantification is desired. However, qualitative quantification can still be reported with a lower number of segments.

### Spreadsheet Simulation

The above calculations can be tested using a spreadsheet simulation. In this simulation, an array of cells equivalent to a total number of viral particles (Nλ) may be generated to randomly return an integer from 1 to the number of wells used to detect (N). A series of cells may count how many individual viral particles were assigned to each well, and these cells may be counted for how many have at least one virus present (x). These simulations may be run several times in parallel to approximate a statistical sample of expected outcomes.

Where the detection limit of each segment is greater than one, Poisson mathematics can be applied to generalize the approach by determining the probability that a specific number of virus particles is present in a given segment, given an average count of virus particles in each segment.

The workflow steps to the complete process to detect virus and bacteria include frost freezing and/or liquid capture, optionally lysing by organic solvent and direct detection without additional sample cleanup. The organic solvent will kill and deactivate the virus so that it is no longer capable of infecting. The workflow of the invention may detect RNA, DNA, chemicals, proteins, carbohydrates, virus, bacteria, spores, and all biomolecules.

Visible or fluorescent detection may be used. For high sensitivity, digital PCR may be used.

Detection may be one sample at a time, or several samples in parallel. In some embodiments, groups are tested.

Sample processing and reporting may be done with an instrument or with a cell phone or smart device.

The detection and reporting may be done with a smart device tied to submitted samples and a subject's phone with identification. The subject's smart device may submit sample with a scan bar code, QR code to tie the sample to a person with reporting mechanism. A “yes” or “no” report can be given along with report giving guidance on distances to be safe. An initial report can be given with LAMP reporting if any highly infectious individuals are present, but LAMP analysis can continue to give yes and no answers at low infectiousness. The technology can quantify the amount of virus, DNA, RNA, bacteria or organic chemical in a room, aircraft or any interior space.

Any smart phone or smart device equipped with a camera, internet connection, and able to run applications can be used for data analysis and reporting. The camera continuously monitors the reaction tubes for changes in color or fluorescence that would indicate a positive result. The application (app) processes the data from the camera, and reports to relevant parties.

The phone camera may continuously monitor 96 vial locations for example and identify them as either a fluorescent tube, a non-fluorescent tube, or an empty well. The app records the time that a new tube is added to the rack, and records the time when fluorescence becomes bright enough to detect. Depending on processing power, the app may quantify brightness over time from individual wells and calculate time of maximum increase in fluorescence. Either time point could estimate viral load in sample. By monitoring for both start and end-time, different samples may be run independently in parallel, with monitoring beginning as soon as each tube is added. At a predefined endpoint, such as one hour without fluorescence, a sample is considered negative. If a sample fluoresces, it is considered positive.

Once a positive or negative result is determined, various pre-defined groups may be automatically informed. The test subject typically receives a message, either through the app or via a text message generated by the app. In order to receive a message, the test subject must input their contact information and indicate informed consent for the test. Other people who were tested at the same site within a given period of time may also be alerted that they may have been exposed. If viral load has been quantified, level of exposure can be estimated. Depending on technical capabilities of location tracking, exposure may be estimated with more precision. For example, a person who has tested may receive a message stating “You appear to have spent 30 minutes within 1 meter of a person shedding 10,000 viral particles per minute. Your risk of infection is 50%”. The test subject may be advised to wear a mask.

Data may also be sent to the organization conducting the test, as well as local health officials. If the test is conducted at a movie theater or airport, the movie theater or airport can be informed so they can take action to protect their patrons and follow sanitization procedures. Airports in particular may further contact airlines, including those operating specific flights to take action depending on whether the subject has already boarded a plane or not. The destination airport may also be contacted in advance, to prepare for potential exposure as the plane unloads. Companies may choose to make testing status of employees publicly accessible on the app. For example, “Steve the cashier tested negative at 10:30 am.”

Data may also be reported to local health authorities or researchers as desired. If tests are widely used and recorded, they may add to the growing body of statistical samples for asymptomatic monitoring.

Test subjects may provide personal contact information as well as consent for reporting at the time of testing. In one instance, a phone number may be used, as it is a unique identifier as well as a convenient means of making contact. Test subjects may also download the app for more detailed information. If a user chooses not to use the app, he or she may receive text notifications regarding his or her test result, as well as notifications if there is a possibility of exposure. If a user chooses to use the app, he or she may access the current status of their test, as well as publicly available testing data. Tests in progress may be expressed in terms of decreasing possible viral load. For example, a high viral load of 40,000 virus may show a positive result after 10 minutes, so at 10 minutes with no positive result, the app can report that the viral load is less than 40,000. As time progresses, this maximum possible viral load will decrease. These numbers can also be expressed in terms of the time and distance that can be safely spent with other people, e.g.: “You are safe to spend 1 hour talking from 6 feet away from someone . . . . You are safe to spend 1 hour standing 3 feet from someone . . . ”.

Because time is both a function of probability of transmission and viral load, “safety distance” may be calculated. As time progresses from the beginning of the test, possible viral load decreases exponentially, if a positive result is not detected. The app may calculate in real time a maximum possible viral load and derive the minimum distance that the subject can safely maintain for that period of time. For example, the app may display in real time “You can safely stand 5 feet or 1.5 meters away from others!” Experts believe that as few as 300 viral particles are sufficient to cause an infection of SARS-COV-2. COVID patients have been recorded as exhaling between 60-25,000 viral particles each minute, leading to a wide range of probabilities for transmission. Probability of transmission from one person to another depends on rate of viral shedding, distance between the infectious person and the subject, time spent in contact, and volume of any room they may occupy together. These factors can be expressed in the following equation:

$$VE = S R \times t ( \frac{1}{{d}^{3}} + \frac{3}{v} )$$

wherein VE is viral exposure (number of viral particles), SR is shed rate (particles per minute), t is exposure time (minutes), d is distance to shed source (decimeters) and v is volume of co-occupied space (liters).

This equation can be used to determine what viral shed rate would be needed to infect another person under given conditions of time, distance, and room volume. For example, a person maintaining two meters distance in a large supermarket for one hour would not infect another person unless they were shedding at least 40,000 viral particles per minute, which would be considered a very high level. In contrast, a person sitting 50 cm from another in a moderately sized church for one hour may pass on their infection if they are shedding only 300 viral particles per minute. A passenger on a long train or airplane trip could spread their infection over the course of 12 hours with a shed rate of only 52 viral particles per minute.

Quantitative studies of viral load, whether sampling from breath, saliva, or nasopharyngeal swab have indicated that viral load tends to peak in the first few days of infection, then quickly falls to a lower level, before tapering off over several days. Presently, the general public has no means to estimate their level of infectiousness, and out of caution are encouraged to remain isolated for 10 to 14 days. A rapid, convenient, affordable, and quantitative test could permit recovering patients to estimate their own level of infectiousness, or screen for asymptomatic spread in large groups.

In some aspects, the present invention may be used to detect diseases in which the infectious agent is exhaled, whether the agents are viral, bacterial, or fungal etc.

Alternatively, in other aspects the present invention may be used as a research tool to develop diagnostics.

### EXAMPLES

### Example 1

A 40 mm square thermoelectric cooler, Peltier module TEC1-12706 was placed hot side down on a 70 mm square aluminum heat sink with cooling fan. A 20×20×15 mm freezing cube of aluminum with a 9.7 mm hole was placed on the cold side of the Peltier module. A round aluminum tube vial with dimensions 20 mm long and outside diameter 9.5 mm with a center hole with inside diameter 7.5 mm was placed into the freezing cube in an upright perpendicular position. A 7.75-inch-long Kraft paper straw with outside diameter of 6 mm was placed into the vial in an upright position. A 3D printed plastic fitting with vent holes kept the straw in the upright position. With this device, breath can be exhaled through the straw and into the internal wall of the cooled vial. Breath vapor and liquid particulates accumulated on the inside wall of the vial and the cleaned breath escaped to the top of the vial in the reverse direction. FIG. 7 shows the configuration of the collection device of the present invention used in the experiment.

In one set of experiments 12 V was applied to the Peltier cooler for 60 seconds. One breath was exhaled through the straw taking 8-12 seconds. The vial was removed and centrifuged for 10 seconds. 5 μL of liquid was coalesced and collected with a 20 μL pipette tip. Some liquid remained in the vial. The collected liquid was placed into a 0.2 mL PCR tube with 15 μL of master mix, solvent lysing solution and primers directed to detect the desired virus. RT-qPCR with a Chai Bio (Santa Clara, CA) 16 well instrument was performed in 40 minutes to detect the presence of virus.

In another experiment, the cooling was applied for 120 seconds, and 1.5 exhaled breaths collected. The experiment was performed 4 times. The vials were centrifuged each time the liquid was coalesced and collected. In two of the experiments 15 μL of breath liquid was collected. In another two experiments, sample sizes of 20 μL of breath were collected.

### Example 2

Two stacked Peltier modules were attached to an aluminum heat sink with cooling fan on the bottom, with an 8 cm tall copper tube with internal diameter of 11 mm attached to a copper plate on the top. The tube was insulated with foam and 2 mL of ethanol were added to the tube. A 3 mL disposable Luer lock syringe, 7 cm long, 1 cm diameter was sealed with a cap and deposited into the tube, causing the ethanol to rise to the rim of the tube around it.

The Peltier modules cooled the tube to approximately −40° C. A 3D printed plastic device for directing breath and inducing turbulence was inserted into the syringe, and breath was directed through for 15, 20, 25, and 30 seconds. After each trial, the syringe was removed from the tube and carefully dried. The plastic breath-directing tube was removed, and a plunger was partially inserted into the syringe. Next the cap was removed and replaced with a 200 μL PCR tube, which does not form an air-tight seal. The plunger was fully depressed, and the syringe was spun in a hand-powered centrifuge to elute all liquid. In these 5 experiments, a range of 40-90 μL of liquid were recovered, ranging from 2.5 μL/second to 3.6 μL/second.

### Example 3

An aluminum tube 2.2 cm in diameter with an internal diameter of 1 cm, 8.8 cm long was cooled on dry ice to −40° C. One end was plugged and 2 mL of ethanol were added to the tube to aid in thermal contact. A 3 mL disposable Luer lock syringe, 7 cm long, 1 cm diameter was sealed with a cap and deposited into the tube, causing the ethanol to rise to the rim of the tube around it. A 3D printed plastic device for directing breath and inducing turbulence was inserted into the syringe, and breath was directed through for 10 seconds. This experiment was repeated 6 times, and liquid yields ranged from 25-44 μL, or 2.5-4.4 μL/second.

### Example 4

In another set of experiments, tubes made of two different metals were compared. A 3 mL disposable Luer lock syringe barrel, 7 cm long, 1 cm diameter was sealed with a cap and deposited into copper tube versus an aluminum tube, both cooled to −30 to −32° C. A 3D printed plastic device for directing breath and inducing turbulence was inserted into the syringe barrel, and breath was directed through for 15, 20, 25 and 30 seconds all using one exhaled breath. The collected volumes of liquid from the frost are shown in Table 4:

TABLE 5
collected volumes for Example 4

| | |
|---|---|
| Copper | Aluminum |

| | Volume | | Volume |
|---|---|---|---|
| Time (s) | collected (μL) | Time (s) | collected (μL) |
| 15 | 41 | 15 | 31 |
| 20 | 51 | 20 | 64 |
| 25 | 89 | 25 | 59 |
| 30 | 85 | 30 | 77 |

The results also show that recovery of frost in the device of the invention is rapid at first and then tapers off as frost is collected. In this experiment, in both cases, sufficient liquid for analysis was recovered in 15 seconds.

### Example 5

This example utilizes a disposable/single use device for collection of exhaled breath liquid particles and vapor in which an endothermic process is used to generate a cooling effect by which breath is collected through condensation/freezing. Sampling often takes less than a minute or even less than 30 seconds to generate enough Exhaled Breath Condensate (EBC) for analysis. As the test subject blows through the mouthpiece, breath is directed against a cooled surface inside the syringe barrel and collection tube. Droplets and vapor from the breath condense on the cold surface. After collection, the condensate may be collected by scraping, draining or centrifuging, and the centrifuge allows rapid collection into a collection vial. This process consistently yields more than 50 μL of EBC sample that is ready for analysis using PCR, RT-PCR, RT-LAMP, RPA, CRISPR, microbial culture, mass spectrometry, or other analytical tools. RPA (Recombinase polymerase amplification) is like LAMP and uses isothermal amplification but is performed at lower temperatures e.g., 30°-45° C.

For this example, the collection device was a 5 ml syringe barrel with a sealed needle attached to its luer end fitting and having a breath tube inlet protruding from its open end. The syringe barrel contains a turbulence inducer. The collection device was placed closed end down into a double paper cup holder containing a coolant mixture consisting of sodium chloride and crushed ice. The paper cup holder consisted of a 12 oz (360 mL) outer cup with an 8 oz (240 mL) inner cup containing approximately 7 oz of the salt ice coolant mixture. The outer cup served to insulate the coolant mixture from the surrounding environment, and in particular from the person in contact with the cup holder. A cover was placed over the top of the cups and the syringe barrel was inserted into the cup holder through an aperture in the center of the cover, thereby positioning a lower portion of the syringe barrel to be surrounded by the coolant mixture.

Two strong exhaled breaths were introduced to the device inlet over a 20 second period. The syringe barrel was then removed from the device, the tube inlet and turbulence inducer removed from the syringe barrel and a syringe plunger placed inside the syringe barrel. The needle seat at the end of the needle was removed and the plunger was pushed into the column. The needle was designed to decrease the dead space in the luer connection.

Approximately 50 μL condensate was removed and mixed with 50 μl of acetonitrile/water. In this example, the acetonitrile/water mixture contained 3 copies (statistically calculated) of a killed COVID virus (ZeptoMetrix). The concentration of acetonitrile was 5% in the total mixture. The mixture was aspirated and then expelled and mixed with LAMP master mix (New England Biolabs) in a 200 μL vial. For colorimetric detection, the vial was placed into block heater (Four E's Scientific) For fluorescence detection the vial was placed into a Chai Bio qPCR thermocycler. Detection of the virus was accomplished in 16 min.

In some embodiments, a syringe plunger may be placed into the syringe to scrape the liquid and consolidate the liquid. In this case, the closed end of the tube is opened to let air escape, or the plunger is modified to allow air to escape along the plunger as it is being placed into the syringe. In some embodiments a convention plunger is used, but the syringe is designed to lower the dead volume in the luer fitting and needle. In some embodiments, a centrifuge is used to collect the liquid in a vial at the end of the tube.

In this example, apparatus and kit components to collect breath samples were supplied as follows:

### Packing List

1. Cooling cups and salt (ice is supplied by the customer)
2. Disposable sampling packet
   a. 5 ml syringe barrel
   b. Turbulence Inducer with mouthpiece inserted into syringe barrel
   c. Collection vial (attached to syringe barrel)
   d. Plastic mouthpiece cover (to be removed before use)

### Initial Apparatus Setup

Set up the coolant cups and mix the ice and water. This can be accomplished by shaking (in the same manner as used in a cocktail mixer).

### Sample Collection

1. Tear open sealed sampling packet, hold syringe by plastic sleeve, and place in the coolant cups
2. Allow syringe barrel with mouth insert to cool for 10 or more seconds before taking breath sample.
3. Have test subject remove sleeve and blow firmly through mouthpiece for 20 seconds (about two deep breaths).
4. Typically, 20 seconds and two complete breaths is adequate to collect 50 μL of sample.
5. Children may need more breaths within the same time frame.
6. Multiple breaths or longer sample times will increase yield of sample.

### Important Guidelines for Breathing Samples

When giving breath sample, blow firmly for as long as is comfortable.
Breathing into the apparatus should be comparable to inflating a balloon or spinning a pinwheel.
The goal is to fully empty the lungs with each breath.
Exhale through mouthpiece only. To inhale, either remove lips from mouthpiece or inhale through nose.
Gentle, tidal breathing results in lower yield of volume.

### Factors Influencing Breath Collection Rate

Temperature-Under normal ambient 20° C. conditions, the collector with sodium chloride salt cools to −20° C. within a couple of minutes. Coolers containing dry ice will cool to lower temperatures. Other salts ice combination can produce lower temperatures.

Time-Volume of EBC collected increases with sampling time. The following volumes were obtained at a starting temperature between −20° C. and −28° C., exhaling one complete breath within each 10 second period:

| Sampling time (seconds) | Expected volume (μL) |
|---|---|
| 10 | 40 |
| 20 | 50 |
| 30 | 70 |
| 40 | 90 |

Number of breaths-Collection rate is associated with the number of breaths sampled. One full breath every 10 seconds is recommended for sample collection. Note that collection volume generally increases with more breaths regardless of the breathing rate, although not in a linear fashion after approximately 50 μL is collected.

Lung capacity-Differences in physiology between test subjects may lead to variation in volume of breath, and therefore volume of EBC collected. If insufficient sample is collected from a given test subject, simply repeat sampling process to collect a larger sample. As warm breath is introduced into the apparatus, a small increase in the cooled copper temperature can be detected. The amount of energy can be quantified and correlated to the amount of breath introduced. In this manner a green light can indicate when the apparatus is cool enough to begin introduction of breath sample. Then, as the breath introduction proceeds, a yellow light can be shown to indicate by a slight increase in temperature of the apparatus. A red light can signify that sample introduction may stop and sufficient, specified sample volume has been collected. The appearance of the red light can be correlated to time, EBC volume, the amount of temperature increases and/or the amount of electrical energy needed to counter the warming of the apparatus by the volume of the warm breath introduced. In this way, sampling between individuals can be standardized. In any case, a minimum sample time with clear breathing instructions will produce sufficient volumes.

### Sample Liquid Collection and Processing

1. Remove syringe from apparatus and place directly into centrifuge. Make note of sample number identification in centrifuge.
2. Ensure that centrifuge is balanced by either loading two samples or one sample and one unused syringe.
3. Close lid and ensure that green power switch is in ON position.
4. Hold black switch in front to activate centrifuge for 10 seconds.
5. Twist vial off the tip of the syringe. Collected liquid may be removed via pipette or stored with caps included.
6. Reagents including acetonitrile and reverse transcriptase, controls and amplification reagents may be added into the tube, pre or post centrifugation. Centrifugation will consolidate and mix all reagents.
   COVID 2 Standard with LAMP Detection after Acetonitrile Treatment
1. 5-20% acetonitrile lysing sample prep with 1-2% final acetonitrile detection concentration
2. Materials: NAT-rol COVID positive control (50 cp/μL), COVID LAMP primers (E and N), acetonitrile, 2× lamp mix, nuclease free water, 50× Fluorescent dye, 50× guanidine hydrochloride (New England Biolabs).
3. Prepare 50 μL of 10% acetonitrile in viral sample. Add 5 μL acetonitrile to 45 μL of sample, mix thoroughly by pipetting. Create 10% acetonitrile control by adding Add 5 μL acetonitrile to 45 μL water.
4. Add master mix, primers, incubate at 65° C. and detect. Positive is bright yellow color.

Samples can be analyzed using LAMP, PCR, or other analytical tools. The EBC treated sample was analyzed by RT-PCR and RT-LAMP. Possible methods include PCR, RT-PCR, RT-LAMP, microbial culture, mass spectrometry, or other analytical tools including digitized nucleic acid amplification methods. Amplification methods may be performed with thermal cycling or isothermal. Some isothermal amplification methods include: NASBA, Nucleic acid sequence-based amplification is a method used to amplify RNA; LAMP, Loop-mediated isothermal amplification is a single tube technique for the amplification of DNA. It uses 4-6 primers, which form loop structures to facilitate subsequent rounds of amplification; HAD, Helicase-dependent amplification uses the double-stranded DNA unwinding activity of a helicase to separate strands for in vitro DNA amplification at constant temperature; RCA, Rolling circle amplification starts from a circular DNA template and a short DNA or RNA primer to form a long single stranded molecule; MDA, Multiple displacement amplification is a technique that initiates when multiple random primers anneal to the DNA template and the polymerase amplifies DNA at constant temperature; RPA, Recombinase polymerase amplification is a low temperature DNA and RNA amplification technique.

### REFERENCES

[1] Heyries K A, Tropini C, Vaninsberghe M, Doolin C, Petriv O I, Singhal A, Leung K, Hughesman C B, Hansen C L, Megapixel digital PCR. Nat Methods. 2011 Jul. 3; 8(8):649-51. doi: 10.1038/nmeth. 1640. PMID: 21725299.

1.-39. (canceled)

40. A method of determining a quantity of a target in a biological sample, the method comprising:
(a) obtaining a biological sample by collecting breath condensate over a sample collection period;
(b) segmenting the biological sample into a plurality of independently analyzable units;
(c) analyzing the segmented biological sample to determine a number of units amongst the plurality of independently analyzable units having at least one copy of a target present therein; and
(d) determining a quantity of the target in the biological sample based on the determined number of units,
wherein the target comprises a nucleic acid, and
wherein analyzing the segmented biological sample comprises performing isothermal amplification of the nucleic acid in each of the plurality of independently analyzable units.

41. The method of claim 40, wherein the sample collection period is less than 5 minutes.

42. The method of claim 40, wherein a volume of the biological sample that is segmented into the plurality of independently analyzable units is equal to or less than 500 μL.

43. The method of claim 40 further comprising:
determining a shed rate based on the determined quantity of the target in the biological sample and a duration of the sample collection period.

44. The method of claim 43 further comprising:
comparing the determined shed rate to a threshold; and
generating a notification if the determined shed rate exceeds the threshold.

45. The method of claim 40, wherein determining the quantity of the target in the biological sample uses the equation
$$N λ = ln ( N / N - x )$$
where N is the number of independently analyzable units comprising the plurality of independently analyzable units, x is the determined number of units, and Nλ is the number of copies of the target across all of the plurality of independently analyzable units.

46. The method of claim 40, wherein determining the quantity of the target in the biological sample comprises:
determining a maximum quantifiable threshold based on total number of the plurality of independently analyzable units; and
if the determined number of units is the total number of independently analyzable units, determining that the quantity of the target in the biological sample is equal to or greater than the maximum quantifiable threshold.

47. The method of claim 40, wherein the biological sample is segmented into less than 5000 independently analyzable units.

48. The method of claim 47, wherein the biological sample is segmented into at least 2 but not more than 1000 independently analyzable units.

49. The method of claim 40, wherein the segmented biological sample is analyzed by any of LAMP, RPA, RT-PCR, LC, LC-MS, GC, GC-MS, or Lateral Antigen.

50. The method of claim 40, wherein segmenting the biological sample comprises depositing each portion of a plurality of portions of the biological sample into a respective well amongst a plurality of wells, each well being a respective one of the plurality of independently analyzable units.

51. The method of claim 50, wherein segmenting the biological sample comprises depositing an equal volume of biological sample into each well of the plurality of wells.

52. The method of claim 50, wherein the volume of each well of the plurality of wells is 5 μL or less.

53. The method of claim 50, wherein each well of the plurality of wells includes an organic solvent stored therein.

54. The method of claim 53, wherein a quantity of the organic solvent stored in each well is 5% or more of the volume of the respective portion of the biological sample deposited in the well.

55. The method of claim 53, wherein the organic solvent is at least one of a water miscible solvent and an aprotic solvent.

56. The method of claim 40, wherein at least half of the biological sample is segmented into the plurality of independently analyzable units.

57. The method of claim 40, wherein the duration of the sample collection period is between 5 seconds and 5 minutes.

58. The method of claim 57, wherein the duration of the sample collection period is between 15 seconds and 45 seconds.

59. The method of claim 40, wherein the duration of the sample collection period is 2 minutes or less.

60. The method of claim 40, wherein the duration of the sample collection period is at least 15 seconds.

61. The method of claim 40, wherein obtaining the biological sample comprises capturing six or fewer exhaled breaths of a user.

62. The method of claim 40, wherein the target comprises lysed biological material.

63. The method of claim 40, wherein the target is a pathogen.

64. The method of claim 40, wherein the biological sample comprises virus, bacteria, yeast, cells, or organic molecules.

65. The method of claim 40, wherein the biological sample comprises virus.

66. The method of claim 66, wherein the virus is selected from the group consisting of a Severe Acute Respiratory Syndrome Corona Virus-2 (SARS-COV-2), a Human Immunodeficiency Virus (HIV), a measles virus, an influenza virus, a Herpes simplex virus, a varicella-zoster virus (VZV), a Respiratory syncytial virus (RSV), an Epstein-Barr virus, a Cytomegalovirus (CMV), a Coronavirus, a Rotavirus, a Hepatitis virus, a human papillomavirus, and a BK virus.

67. The method of claim 64, wherein the bacteria is tuberculosis (TB) or staphylococcus.

68. The method of claim 40, wherein obtaining the biological sample comprises:
receiving breath exhaled by a user in a breath collection device; and
cooling the exhaled breath to form the breath condensate containing the biological sample.

69. The method of claim 68, wherein cooling the exhaled breath includes contacting the exhaled breath with a capture surface, the capture surface being cooled by a coolant to cool the exhaled breath, and wherein the method further comprises:
inducing turbulence in flow of breath to enhance contact between the capture surface and the exhaled breath.

70. The method of claim 40 further comprising:
outputting a report of the quantity of the target in the biological sample.

71. The method of claim 70, wherein the output report comprises a quantitative or qualitative indication of a shed rate of pathogen in the biological sample.

72. The method of claim 70, wherein the output report indicates if the target is present in an amount below a detection limit.

73. The method of claim 40 further comprising:
mixing the biological sample with one or more detection reagents before analysis.

74. A method of determining a quantity of a target in a biological sample, the method comprising:
(a) obtaining a biological sample by collecting breath condensate over a sample collection period;
(b) segmenting the biological sample into a plurality of independently analyzable units;
(c) analyzing the segmented biological sample to determine a number of units amongst the plurality of independently analyzable units having at least one copy of a target present therein; and
(d) determining a quantity of the target in the biological sample based on the determined number of units,
wherein the sample collection period is less than 5 minutes.

75. The method of claim 74, wherein the biological sample comprises nucleic acids, and wherein analyzing the segmented biological sample comprises detecting nucleic acid amplification at each of the independently analyzable units.

76. The method of claim 75, wherein the nucleic acid amplification comprises isothermal nucleic acid amplification or PCR.

77. A method of determining a quantity of a target in a biological sample, the method comprising:
(a) obtaining a biological sample by collecting breath condensate over a sample collection period;
(b) segmenting the biological sample into a plurality of independently analyzable units;
(c) analyzing the segmented biological sample to determine a number of units amongst the plurality of independently analyzable units having at least one copy of a target present therein; and
(d) determining a quantity of the target in the biological sample based on the determined number of units,
wherein a volume of the biological sample that is segmented into the plurality of independently analyzable units is equal to or less than 500 μL.
